(19) United States
(12) Patent Application Publication
(10) Pub. No.: US 2025/0127167 A1
(43) Pub. Date: Apr. 24, 2025

(54) ANTIMICROBIAL PEPTIDES

(71) Applicant: YISSUM RESEARCH DEVELOPMENT COMPANY OF THE HEBREW UNIVERSITY OF JERUSALEM LTD, Jerusalem (IL)

(72) Inventor: Meital RECHES, Kfar Bin Nun (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/691,495

(22) PCT Filed: Sep. 14, 2022
(86) PCT No.: PCT/IL2022/050994

§ 371 Date: Mar. 13, 2024

Related U.S. Application Data
(60) Provisional application No. 63/261,204, filed on Sep. 15, 2021.

(51) Int. Cl. A01N 37/46; A01N 25/02; A01P 1/00; C07K 5/06; C07K 5/065

(52) U.S. Cl. CPC A01N 37/46; A01N 25/02; A01P 1/00; C07K 5/06078; C07K 5/06191

(57) ABSTRACT

The invention generally discloses short and ultrashort peptides with improved antimicrobial properties.

### TECHNOLOGICAL FIELD

The invention generally contemplates peptides and uses thereof as antimicrobial agents.

### BACKGROUND OF THE INVENTION

Infectious diseases severely affect all aspects of human life worldwide and have become a serious public health and economic concern. Nearly one-third of these infections are ascribed to viruses. To date, more than 200 infectious diseases have been discovered; some of them such as influenza A virus subtype H1N1, and H5N1, Ebola, human immunodeficiency viruses (HIV), severe acute respiratory syndrome (SARS), and novel coronavirus disease (COVID-19) lead to high morbidity and mortality levels. In particular, the extraordinary epidemic outbreak of the COVID-19 global pandemic created dramatic challenges to human health and well-being; it adversely affected the world economy and threatened the welfare of populations. The COVID-19 pandemic “wake-up call” has motivated researchers to develop novel strategies and methods to overcoming the spread of viruses. Coronavirus can be easily transmitted by touching surfaces contaminated by it and then touching the face (e.g., the eyes, nose, and mouth), which is considered to be one of the major spread routes.

Moreover, unfortunately, most viruses can survive on surfaces composed of metals, oxides, and polymers for a long time (from tens of hours to several days). For example, it has been reported that the COVID-19 viral pathogens can attach to surfaces and survive for 4 days on glass and ˜7 days on stainless steel and polymeric surfaces. Although some viruses (e.g., HIV) cannot spread outside the body, others, respiratory infection viruses, can easily attach to the surface and remain adherent, which increases the risk of infection. Although direct physical or chemical sanitization by spraying or wiping with disinfectants (such as hydrogen peroxide or rubbing alcohol) is effective to terminate the fomite transmissions through surface touching, the effect of a virus-free environment is not permanent. A better, more effective strategy is to provide the surface with an antiviral activity that will eliminate the viruses quickly after their attachment. Therefore, there is an urgent need to design and develop new, innovative antiviral surfaces and coatings that could be widely applied to various substrates to lower the risk of viral transmission and harm to public health.

Several reports show that some natural extracts (e.g., phenolic compounds, essential oils, and polysaccharides) can be incorporated into coatings to provide antiviral properties. For example, tea tree and eucalyptus oil coatings exhibited good antiviral activity against the influenza virus. However, integrating and applying these oils as effective coatings are somewhat challenging. Another proposed strategy is to incorporate metal nanomaterials such as copper, silver, and zinc, which act as antiviral agents. Inspired by the idea of using nanomaterial-based antiviral agents, researchers have also developed polymeric and organic antiviral coatings. For example, polyethyleneimine-coated glass slides were shown to decrease bacteriophage PRD1 virus titers compared with bare glass. However, the toxicity of both organic and inorganic nanoparticles may cause health and environmental issues. To overcome this problem, researchers have been searching for environmentally friendly and low-toxicity antiviral coatings.

Peptides have also been suggested as antiviral compounds. A 20-amino acid peptide derived from a signal sequence of fibroblast exhibited a broad-spectrum antiviral activity against influenza viruses including the H5N1 subtype. They suggested that the peptide is attached to a cellular receptor and that this mechanism prevents viral infection. In addition, several antimicrobial peptides (AMPs) have been tested as antiviral agents against SARS-CoV-2 and other RNA-based viruses due to their low toxicity. Although significant efforts have been made to develop antiviral peptides as therapeutic agents against viral infections, it would be beneficial to utilize them as antiviral coatings that block the contact transmission of the virus, especially respiratory tract viruses.

Self-assembled peptides can serve as good candidates for fabricating functional coatings. The inventors of the present technology have previously shown that the tripeptide DOPA-Phe(4F)-Phe(4F)—OMe can self-assemble (via the DOPA entity that has “sticky” properties) into a coating that prevents the adhesion of proteins and bacteria on surfaces via the F moiety that has antiadherence characteristics. In addition, they have recently reported that this peptide self-assembles in an aqueous solution into spherical particles. Despite its antuadherence properties, F-based peptides are not antimicrobial [1].

### BACKGROUND PUBLICATIONS

[1] Nir, S.; Zanuy, D.; Zada, T.; Agazani, O.; Aleman, C.; Shalev, D. E.; Reches, M. Tailoring the Self-Assembly of a Tripeptide for the Formation of Antimicrobial Surfaces. Nanoscale 2019, 11 (18), 8752-8759

### GENERAL DESCRIPTION

The inventors of the technology disclosed herein have now demonstrated that peptides that lack the antiadherence fluoro atom (F moiety), such as DOPA-Phe(4Br)-Phe(4Br)—OMe and DOPA-Phe-Phe-OMe exhibit antimicrobial and in particular antiviral properties, both in solution and as solid films on various surfaces. As demonstrated, these peptides reduce the number of T4 bacteriophage and canine coronavirus cases by more than 99.9%, opening the door for extensive full spectrum applications in humans, animals and in agricultural uses.

The elimination of an antifouling moiety, such as an F moiety, from short and even ultrashort peptides, has unexpectedly demonstrated increased antimicrobial properties. This trend is highly surprising particularly in view of the fact that the antimicrobial properties were observed also in cases where the peptide did not form into solid films on a surface. Solutions of the F-less peptides were found highly antimicrobial, suggesting that film forming properties are not essential for achieving effective and prolonged antimicrobial properties.

In a first aspect of the invention, there is provided an antimicrobial formulation comprising at least one antimicrobial peptide of structure (I): DOPA-X, wherein X is an amino acid or a peptide comprising between 2 and 4 amino acids, wherein the peptide of structure (I) is free of F atoms or free of antifouling moieties or atoms.

The antimicrobial peptide of structure (I) is a short or an ultrashort peptide comprising overall between 2 and 5 amino acids, one of which being DOPA, as defined herein, and the other being selected amongst aromatic amino acids.

In some embodiments, the antimicrobial peptide is a short peptide of structure (I), wherein X comprises 2 or 3 or 4 amino acids. In some embodiments, the antimicrobial peptide is an ultrashort peptide of the structure (I), wherein X is a single amino acid.

In some embodiments, the antimicrobial peptide of structure (I) is of a structure (II): DOPA-(AA)ₙ-M, wherein
DOPA is 3,4-dihydroxy-L-phenylalanin (DOPA), or a hydroxylated DOPA, such as hydroxy-DOPA, dihydroxy-DOPA and trihydroxy-DOPA;
AA is an amino acid or an amino acid sequence or peptide comprising between 2 and 4 amino acids;
n is an integer between 1 and 4; and
M is a functionality or a group of atoms (being a terminal group of the end-of chain amino acid in the sequence), M may be:
   (1) —O-M, wherein —O— is an oxygen atom of the terminal amino acid and M is selected from a metal cation (such as Li, Na, K, and other monovalent metal cations); —H; —C₁-C₅alkyl (such as methyl, ethyl, propyl, butyl, pentyl, isopropyl, ter-butyl and others); —C₆-C₁₀aryl (such as phenyl and naphthyl); —C(═O)—O—C₁-C₅alkyl (wherein the alkyl is methyl, ethyl, propyl, butyl, pentyl, isopropyl, ter-butyl and others); —C(═O)—O—C₆-C₁₀aryl (wherein the aryl is phenyl or naphthyl); —C(═O)—O—C₁-C₅alkylene-C₆-C₁₀aryl (such as benzyloxycarbonyl, Cbz); —NH₂; —NHR₁, —NHR₁R₂, —NR₁R₂R₃, wherein each of R₁, R₂ and R₃, independently of the other is —H or a —C₁-C₅alkyl (such as methyl, ethyl, propyl, butyl, pentyl, isopropyl, ter-butyl and others); or
   (2) an amine or an ammonium (being an amide form of the terminal amino acid group) selected from —NH₂; —NHR₁, —NHR₁R₂, —NR₁R₂R₃, wherein each of R₁, R₂ and R₃, independently of the other is —H or a —C₁-C₅alkyl (such as methyl, ethyl, propyl, butyl, pentyl, isopropyl, ter-butyl and others).

In some embodiments, the peptide of structure (II) is DOPA-(AA)ₙ-O-M (being an ester or an equivalent form of the peptide), wherein each of DOPA, AA, n, O and M is as defined herein.

In other words, a peptide of structure (II) may be generally depicted to be of the short representation DOPA-( . . . )—C(═O)—OM or DOPA-( . . . )—C(═O)—N— . . .

In some embodiments, the peptide of structure (II) is DOPA-(AA)ₙ-NH₂, DOPA-(AA)ₙ-NHR₁, DOPA-(AA)ₙ-NHR₁R₂⁺, or DOPA-(AA)ₙ-NR₁R₂R₃⁺ (each of which being an amide form of the peptide), wherein each of R₁, R₂ and R₃, independently of the other is —H or a —C₁-C₅alkyl, and wherein the nitrogen atom is positively charged, the peptide is associated with at least one counter ion. The counter ion may be any single atom anion or an anionic group of atoms. Such counter ions may be a halide (a chloride, a bromide, an iodide), a sulfate, a pyrosulfate, a bisulfate, a sulfite, a bisulfite, a nitrate, a phosphate, a monohydrogen phosphate, a dihydrogen phosphate, a metaphosphate, a pyrophosphate, an acetate, a propionate, a caprylate, an isobutyrate, an oxalate, a malonate, a succinate, a suberate, a sebacate, a fumarate, a maleate, a mandelate, a benzoate, a chlorobenzoate, a methylbenzoate, a dinitrobenzoate, a phthalate, a benzenesulfonate, a toluenesulfonate, a phenylacetate, a citrate, a lactate, a maleate, a tartrate, a methanesulfonate and others.

In some embodiments, each of the amino acids designated AA is an aromatic amino acid. In some embodiments, at least one of the amino acids designated AA is an aromatic amino acid. In some embodiments, at least one of the amino acids designated AA is a brominated or a chlorinated aromatic amino acid.

In some embodiments, the aromatic amino acid is selected from phenylalanine, tryptophan and tyrosine. In some embodiments, the aromatic amino acid is phenylalanine or a derivative thereof.

In some embodiments, the phenylalanine derivatives is 4-methoxy-phenylalanine, 4-carbamimidoyl-1-phenylalanine, 4-chloro-phenylalanine, 3-cyano-phenylalanine, 4-bromo-phenylalanine, 4-cyano-phenylalanine, 4-hydroxymethyl-phenylalanine, 4-methyl-phenylalanine, 1-naphthyl-alanine, 3-(9-anthryl)-alanine, 3-methyl-phenylalanine, m-amidinophenyl-3-alanine, phenylserine, benzylcysteine, 4,4-biphenylalanine, 2-cyano-phenylalanine, 2,4-dichloro-phenylalanine, 3,4-dichloro-phenylalanine, 2-chloro-penylalanine, 3,4-dihydroxy-phenylalanine, 3,5-dibromotyrosine, 3,3-diphenylalanine, 3-ethyl-phenylalanine, 3-chloro-phenylalanine, 3-chloro-phenylalanine, 4-amino-L-phenylalanine, homophenylalanine, 3-(8-hydroxyquinolin-3-yl)-1-alanine, 3-iodo-tyrosine, kynurenine, 3,4-dimethyl-phenylalanine, 2-methyl-phenylalanine, m-tyrosine, 2-naphthyl-alanine, 5-hydroxy-1-naphthalene, 6-hydroxy-2-naphthalene, meta-nitro-tyrosine, (beta)-beta-hydroxy-1-tyrosine, (beta)-3-chloro-beta-hydroxy-1-tyrosine, o-tyrosine, 4-benzoyl-phenylalanine, 3-(2-pyridyl)-alanine, 3-(3-pyridyl)-alanine, 3-(4-pyridyl)-alanine, 3-(2-quinolyl)-alanine, 3-(3-quinolyl)-alanine, 3-(4-quinolyl)-alanine, 3-(5-quinolyl)-alanine, 3-(6-quinolyl)-alanine, 3-(2-quinoxalyl)-alanine, styrylalanine, 4-iodo-phenylalanine, 4-nitro-phenylalanine, phosphotyrosine, 4-tert-butyl-phenylalanine, 3-amino-L-tyrosine, 3,5-diiodotyrosine, 3-amino-6-hydroxy-tyrosine, and/or tyrosine.

In some embodiments, the aromatic amino acid is brominated, e.g., comprises a Br atom, or chlorinated, e.g., comprises a Cl atom, on the aryl functionality (phenyl), at any position of the aromatic ring. In some embodiments, the Br or Cl atom is positioned at the or to (2Br or 2Cl), meta (3Br or 3Cl) or para (4Br or 4Cl) position. In some embodiments, the brominated or chlorinated aromatic amino acid is a brominated or chlorinated phenyl alanine (4Br or 4Cl).

In some embodiments, the aromatic acid is phenyl alanine (Phe) or a brominated or chlorinated phenylalanine (Phe(Br) or Phe(Cl), respectively). In some embodiments, the aromatic amino acid is a brominated phenyl alanine Phe(4Br).

In some embodiments, in a peptide of structure (II), n is 1 or 2. In some embodiments, in a peptide of structure (II), wherein n is 1 or 2, one or both of the amino acids is an aromatic amino acid; or one or both of the amino acids is a brominated amino acid.

In some embodiments, the antimicrobial peptide used according to the invention is selected from:
DOPA-(AA)n-O-M, wherein each of AA, n, O and M is as defined herein;
DOPA-(AA)n-NH₂, DOPA-(AA)n-NHR₁, DOPA-(AA)n-NHR₁R₂⁺, or DOPA-(AA)n-NR₁R₂R₃⁺, wherein each of R₁, R₂ and R₃, independently of the other is —H or a —C₁-C₅alkyl;
DOPA-Phe-Phe-OM, wherein M is as defined herein;
DOPA-Phe-Phe(4Br)-OM, wherein M is as defined herein;
DOPA-Phe(4Br)-Phe-OM, wherein M is as defined herein;
DOPA-Phe(4Br)-Phe(4Br)-OM, wherein M is as defined herein;
DOPA-Phe-OM, wherein M is as defined herein;
DOPA-Phe-Phe(4Cl)-OM, wherein M is as defined herein;
DOPA-Phe(4Cl)-Phe-OM, wherein M is as defined herein;
DOPA-Phe(4Cl)-Phe(4Br)-OM, wherein M is as defined herein;
DOPA-Phe(4Cl)-Phe(4Cl)-OM, wherein M is as defined herein;
DOPA-Phe(4Br)-Phe(4Cl)-OM, wherein M is as defined herein;
DOPA-Phe-OM, wherein M is as defined herein;
DOPA-Phe-NH₂;
DOPA-Phe(4Br)—NH₂;
DOPA-Phe(4Cl)—NH₂;
DOPA-Phe-Phe-OMe (OMe=O-methyl);
DOPA-Phe-Phe(4Br)—OMe;
DOPA-Phe-Phe(4Cl)—OMe;
DOPA-Phe(4Br)-Phe-OMe;
DOPA-Phe(4Br)-Phe(4Br)—OMe;
DOPA-Phe(4Cl)-Phe-OMe;
DOPA-Phe(4Cl)-Phe(4Br)—OMe;
DOPA-Phe(4Cl)-Phe(4Cl)—OMe;
DOPA-Phe(4Br)-Phe(4Br)—OMe;
DOPA-Phe-OMe; and
Cbz-DOPA-Phe-OH.

Formulations of the invention may be designated antimicrobial formulations, namely having themselves antimicrobial properties, or as formulations for forming antimicrobial films on a surface region, or as formulations for preventing attack or damage or degradation or decomposition or poisoning due to presence of a microbial infection source, or as formulations for forming protective antimicrobial films or coats, or as formulations for agricultural uses for applications on live plants, on fruits and vegetables or seeds.

In some embodiments, formulations of the invention may be formed as agricultural formulations for decreasing microbial load in a medium which may be a liquid medium or a surface. The decrease in the microbial load may be by preventing microbial infection, propagation, attachment or spreading; by eradicating (decrease number) of microbial cells or virions in a target (surface, bulk solution), after they have already been established; or by repelling microbial settling or attachment or assembly on a surface of an agricultural product such as live plants, pre-harvested or post-harvested fruits, vegetables, flowers and seeds, for improving their growth, storage, handling, safety, effectiveness and for preventing spoilage and production of microorganism-derived undesirable by-products, such as carbon dioxide, methane, nitrogenous compounds, butyric acid, propionic acid, lactic acid, formic acid, sulfur compounds, and other gases and acids that can have a detrimental effect. In some embodiments, the formulation of the invention prevents propagation and spreading of the microorganism by prevention of assembly and production of new microbial cells or viral capsids.

In some embodiments, formulations or peptides of the invention may be formulated or used as disinfectant compositions or as preservatives. When used as preservatives, the peptides can be incorporated into any suitable product, such as a paint, a latex emulsion, a polymer emulsion, an adhesive, a sealant, a caulk, a mineral or pigment slurry, a printing ink, a pesticide formulation, a household product, a personal care product, a hygiene product, a metal working fluid, a pharmaceutical, a foodstuff, a food additive, any packaging material and the like.

Personal care products that may contain the peptide(s) or topical pharmaceutical formulations which may include an effective amount of a peptide according to the invention may include an emulsion, a cream, a toner, an essence, a pack, a gel, a powder, a makeup base, a foundation, a lotion, an ointment, a patch, a cosmetic solution, a cleansing foam, a cleansing cream, a cleansing water, a body lotion, a body cream, a body oil, a body essence, a shampoo, a rinse, a body cleanser, a soap, a hair dye, a spray, etc.

In some embodiments, formulations or peptides of the invention may be formed into films or coated on a surface region of an object, such as aesthetic objects, cosmetic objects, medical devices, medical surfaces, etc. The antimicrobial coating formed by peptides or formulations of the invention may be for use as a long-term coating, for example, more than 1 month, on a surface of consumer goods containers, means of transport, furnishing objects, common spaces, equipment, clothing, surfaces of medical devices (prostheses, catheters, bandages, and others), or any other surface which favors proliferation of bacterial colonies and the adhesion of viral particles.

Depending on the type of formulation and the intended use, the formulation may comprise one or more active or inactive (or inert) additives or materials. Formulations of the invention may comprise a carrier such as water or other aqueous media, stabilizers, antioxidizing agents, salts, desiccants, defoliants, surfactants, coloring agents, emulsifiers, dispersants, metals such as copper, essential oils, drugs as well as active agents which may be selected and tailored for a specific use.

In some embodiments, formulations of the invention are aqueous formulations, optionally comprising an alcohol.

In some embodiments, formulations of the invention are dispersions, suspensions emulsions, optionally comprising water.

Formulations of the invention may be formed into any type of liquid formulation or a solid formulation, e.g., a solid formulation which can be redispersed or reconstituted in a fluid. The formulations may be formed into aerosol formulations, liquid formulations, spray formulations, dust formulations, dry flowable formulations, granulated formulations, wettable formulations, brushing formulations, polymeric formulations and others.

In some embodiments, the peptide utilized in formulations of the invention is provided in a soluble, dispersed or suspended form. The peptides can also be provided as in dispersible solid forms capable of redissolution or redispersion in a liquid medium, e.g., an aqueous medium.

In some embodiments, the peptide utilized in formulations of the invention is provided in a particulate form or an encapsulated form or present or held within a solid matrix which may be a porous matrix, a soluble matrix, a metallic matrix, a polymeric matrix or any matrix known in the art. In some embodiments, the peptide is encapsulated or held within a solid matrix such as capsules or porous solid materials and may be used as such, wherein optionally the peptide is contained to leech out from the capsule or solid matrix over a period of time, and optionally at a predetermined rate. The delivery profile may be tailored by means known in the art.

The amount of the peptide in a formulation according to the invention may vary based, inter alia, on the type of peptide(s) used, the intended application, the desired effect, etc. Generally speaking, the peptide may be present in an amount ranging between 0.01 and 20 wt %, or between 0.5 and 10 wt %, or between 1 and 5 wt %.

As states herein, in an antimicrobial formulation or combination comprising at least one antimicrobial peptide of the structure (I), as defined, the peptide of structure (I) is free of F atoms or free of any antifouling moiety or atom. In other words, the peptide of structure (I) does not have any group that comprises an F atom, nor any group that can be defined as antifouling or which is defined as an antiadherence group.

The antifouling groups excluded from peptides of structure (I) are those capable, or known from the scientific literature to prevent and control fouling of a surface of a solid composition or an object by minimizing, diminishing, or preventing adhesion of bacteria, viruses, and/or fungi. Examples of such groups include F atoms or groups containing one or more F atoms, each of which being individually excluded from peptides of structure (I), as disclosed herein.

Unlike antifouling peptides, which work in a “Teflon-like” mechanism by preventing adhesion due to the presence of F moieties on the peptide, peptides of the invention exhibit antimicrobial properties which are unique and evident not only when the peptides are formed into a film on a surface region of an object, but more interestingly in solution. When peptides disclosed herein are added into a medium, be it a liquid medium or a solid medium, the peptides are capable of rendering the medium microbe-free by directly interacting with microbes and especially the membranes of the microbes that may be present in the media. Without wishing to rely on mechanistic or theoretical discussion, it is believed that the peptides interact with the microbes to interfere with their ability to assemble and produce transmittable bodies such as virions or new cells. It is further believed that the interaction involves peptide penetration through the microorganism's membrane. Thus, peptides used according to the invention induce not only protection against microorganisms but also eradicate microorganisms present, providing both prevention and eradication (or treatment) modalities also in a liquid medium. Peptides used according to the invention may therefore be considered antibacterial, antifungal, antimycotic, antiparasitic, antiprotozoal, antiviral, antiinfectious, antiinfective and/or germicidal, algicidal, amoebicidal, microbicidal, bactericidal, fungicidal, parasiticidal, protozoacidal, or protozoicidal.

While antifouling materials prevent the adhesion of microorganisms to the surface, compounds of the invention kill, disassemble or prevent new assembly of microorganisms and prevent their proliferations, rendering them ineffective. While antifouling materials may be tested as effective when assembled on a surface of an object, the antimicrobial peptides of the invention are effective in solution or in any other medium, thus presenting a more effective and versatile platform for achieving microbe free media.

In some embodiments, peptides used according to the invention exhibit antiviral, antibacterial and antifungal properties.

Peptides used according to the invention enable inter alia eliminating or decreasing proliferation of existing microorganisms in a target medium or surface and prevention of biofilm generation, formation or growth,

Thus, the term “antimicrobial” encompasses prevention and/or retardation of growth, and/or prevention of accumulation of microorganisms, and or decrease in the number of viable viruses and infective virons, bacteria, undulating bacteria, spirochetes, spores, spore-forming organisms, gram-negative organisms, gram-positive organisms, yeasts, fungi, molds, aerobic organisms, anaerobic organisms and mycobacteria.

Non-limiting examples of microbial organisms that can be controlled using formulations of the invention include
bacteria from the genus Aeromonas (e.g. A. hydrophilia), Arcobacter, Bacillus (e.g. B. cereus), Brochothrix (e.g. B. thermosphacta), Campylobacter (e.g. C. jejuni), Carnobacterium (e.g. C. piscicola), Clostridium (e.g. C. perfringens, C botulinum), Enterobacteriacae, Escherichia (e.g. E. coli), Listeria (e.g. L. monocytogenes), Pseudomonas (e.g. P. putida, P. fluorescens), Salmonella (e.g. S. Typhimurium), Serratia (e.g. S. liquefaciens), Shigella, Staphylococcus (e.g. S. aureus), Vibrio (e.g. V. parahaemolyticus, V. cholerae) and Yersinia (e.g. Y. enterocolitica); Erwinia, Pseudomonas pyocyanea, and Corynebacterium xerosis,
fungi such as Aspergillus flavum and Penicillium chrysogenum; parasites such as Entamoeba (Entamoeba histolytica), Balantidium (Balantidium cob), Cryptosporidium (e.g., Cryptosporidium parvum), Cyclospora (e.g. Cyclospora cayetanensis), Giardia (e.g. Giardia lamblia, Giardia intestinalis), Isospora (Isospora belle), Microsporidia (Enterocytozoon bieneusi, Septata intestinalis), Trichinella spiralis and Toxoplasma gondii; Fusarium oxysporum, Penicillium italicum, Colletotrichum gloeosporioides, Colletotrichum capsica, and Fusarium solani, Pythium, Pythium sp., Sclerotium rolfsii.
viruses and infective viron of viruses selected from bacteriophages, coronaviridae/corona-virus, orthomyxoviridae, paramyxoviridae, Coxsackie family of viruses and adenoviridae family; including corona-virus, such as a COVID-19 causing pathogen, e.g., SARS-CoV-2, encompassing SARS-CoV-2 having mutations that may be found in the entire genome of SARS-CoV-2 strains, e.g., in the 5′ UTR, ORF1ab polyprotein, intergenic region, envelope protein, matrix protein and nucleocapsid protein, Tobamovirus, Tomato brown rugose fruit virus.

In some embodiments, formulations of the invention are tailored for use as antiviral formulations.

In some embodiments, the antiviral formulations are effective against bacteriophages and viral infectors both in human and non-human animals.

In some embodiments, the viral infector is a human or a canine coronavirus.

In some embodiments, the antimicrobial formulations of the invention are further used against vegetative or dormant forms of bacteria and fungi, such as spores wherein their growth cycle may be controlled using formulations and methods disclosed herein.

The invention further provides use of at least one antimicrobial peptide of structure (I) or (II), as defined herein, as an antimicrobial agent or as an agent capable of rendering antimicrobial properties to an object, to a formulation or to a combination of materials.

The invention further provides use of at least one antimicrobial peptide of structure (I) or (II), as defined herein, in a method of eradicating or reducing microbial population in a solid or liquid medium.

The invention further provides use of at least one antimicrobial peptide of structure (I) or (II), as defined herein, in a method of preparing an antimicrobial formulation, object or surface.

The invention further provides a film comprising a peptide of structure (I) or (II), or a film formed of a formulation comprising a peptide of structure (I) or (II).

Further provided are particles formed of a peptide of structure (I) or (II), wherein said particles are optionally in a form of capsules. The particles may be spherical in shape and may be characterized by surface pores forming an internal or surface volumes or cavities which may or may not contain a component of the formulation in which the particles are formed or contained.

The invention further provides a method of eradicating or reducing a population of microorganism in a solid or liquid medium, the method comprises contacting or adding or treating or allowing interaction of said medium with a peptide of structure (I) or (II) or a formulation comprising same.

As used herein, the expression “eradicating or reducing population” refers to the ability of peptides disclosed herein to reduce a population of or propagation of or to increase ineffectiveness of viruses including infective virons, bacteria, undulating bacteria, spirochetes, spores, spore-forming organisms, gram-negative organisms, gram-positive organisms, yeasts, fungi, molds, aerobic organisms, anaerobic organisms and/or mycobacteria by 10, 20, 30, 40, 50, 60, 70, 80, or 90% or more. In some embodiments, the population is eradicated, namely at least 95, 96, 97, 98 or 99% or 100% of the population is rendered non-viable, non-propagating or non-infective or dead.

The invention further provides a method of inducing or endowing antimicrobial properties to a surface region of an object, the method comprises contacting said surface region with a peptide of structure (I) or (II) or a formulation comprising same and optionally allowing said formulation to form a solid film material of said peptide.

The invention further provides a method of reducing microbial load on or in an object, the method comprising contacting said object with a peptide of structure (I) or (II) or a formulation comprising same and optionally allowing said formulation to form a solid film material of said peptide

The invention further provides a method for protecting live plants, post harvest plants or their parts, fruits, vegetables, seeds, seedings from attack by at least one microbial source or pathogen, the method comprising contacting said live plants, post harvest plants or their parts, fruits, vegetables, seeds, seedings with a peptide of structure (I) or (II) or a formulation comprising same and optionally allowing said formulation to form a solid film material of said peptide.

Further provided is a method of protecting plants including fruits or vegetables, pre- or post-harvesting from attack by a microbial source or a pathogen, the method comprising contacting said fruits or vegetables with a peptide of structure (I) or (II) or a formulation comprising same and optionally allowing said formulation to form a solid film material of said peptide.

The invention further provides a method of protecting seeds against microbial attack by a microbial source or a pathogen, the method comprising contacting said fruits or vegetables with a peptide of structure (I) or (II) or a formulation comprising same and optionally allowing said formulation to form a solid film material of said peptide.

In some embodiments, the contacting may be achievable by any known application method, including spraying, brushing, washing, coating, printing, immersing, dipping or provided via irrigation or sprinkling etc.

Further provided is a kit comprising a formulation of the invention in a mixed or pre-mixed form and instructions of use.

In some embodiments, the kit comprises an amount of a peptide as disclosed herein and a liquid carrier suitable for making the formulation.

In some embodiments, the kit comprises an amount of a peptide as disclosed herein dissolved, suspended or dispersed in liquid carrier.

The invention thus further provides:

An antimicrobial formulation comprising at least one antimicrobial peptide of structure (I): DOPA-X, wherein X is an amino acid or a peptide comprising between 2 and 4 amino acids, wherein the peptide of structure (I) is free of F atoms or free of antifouling moieties or atoms.

In some configurations of the invention, the peptide of structure (I) is a peptide comprising between 2 and 5 amino acids, one of which being the DOPA, and the other amino acids being selected amongst aromatic amino acids.

In some configurations of the invention, the peptide is a peptide of structure (I), wherein X comprises 2 or 3 or 4 amino acids.

In some configurations of the invention, X is a single amino acid.

In some configurations of the invention, the peptide of structure (I) is of a structure (II): DOPA-(AA)ₙ-M, wherein
DOPA is 3,4-dihydroxy-L-phenylalanin (DOPA), or a hydroxylated DOPA,
AA is an amino acid or an amino acid sequence or peptide comprising between 2 and 4 amino acids;
n is an integer between 1 and 4; and
M is a functionality or a group of atoms present at the terminal end of the peptide, M being:
   (1) —O-M, wherein —O— is an oxygen atom of the amino acid and M is selected from a metal cation (such as Li, Na, K, and other monovalent metal cations); —H; —C₁-C₅alkyl; —C₆-C₁₀aryl; —C(═O)—O—C₁-C₅alkyl; —C(═O)—O—C₆-C₁₀aryl; —C(═O)—O—C₁-C₅alkylene-C₆-C₁₀aryl; —NH₂; —NHR₁, —NHR₁R₂, —NR₁R₂R₃, wherein each of R₁, R₂ and R₃, independently of the other is —H or a —C₁-C₅alkyl; or
   (2) and amine or an ammonium selected from —NH₂; —NHR₁, —NHR₁R₂, —NR₁R₂R₃, wherein each of R₁, R₂ and R₃, independently of the other is —H or a —C₁-C₅alkyl.

In some configurations of the invention, the hydroxylated DOPA is hydroxy-DOPA, dihydroxy-DOPA or trihydroxy-DOPA.

In some configurations of the invention, the peptide of structure (II) is DOPA-(AA)ₙ-O-M, wherein each of DOPA, AA, n, O and M is as defined herein.

In some configurations of the invention, the peptide of structure (II) is DOPA-(AA)ₙ-NH₂, DOPA-(AA)ₙ-NHR₁, DOPA-(AA)ₙ-NHR₁R₂⁺, or DOPA-(AA)ₙ-NR₁R₂R₃+, wherein each of R₁, R₂ and R₃, independently of the other is —H or a —C₁-C₅alkyl, and when the nitrogen atom is positively charged, the peptide is associated with at least one counter ion.

In some configurations of the invention, each of the amino acids designated AA is an aromatic amino acid.

In some configurations of the invention, at least one of the amino acids designated AA is an aromatic amino acid.

In some configurations of the invention, at least one of the amino acids designated AA is a brominated or a chlorinated aromatic amino acid.

In some configurations of the invention, the aromatic amino acid is selected from phenylalanine, tryptophan and tyrosine.

In some configurations of the invention, the aromatic amino acid is phenylalanine or a derivative thereof.

In some configurations of the invention, the phenylalanine derivatives is 4-methoxy-phenylalanine, 4-carbamimidoyl-1-phenylalanine, 4-chloro-phenylalanine, 3-cyano-phenylalanine, 4-bromo-phenylalanine, 4-cyano-phenylalanine, 4-hydroxymethyl-phenylalanine, 4-methyl-phenylalanine, 1-naphthyl-alanine, 3-(9-anthryl)-alanine, 3-methyl-phenylalanine, m-amidinophenyl-3-alanine, phenylserine, benzylcysteine, 4,4-biphenylalanine, 2-cyano-phenylalanine, 2,4-dichloro-phenylalanine, 3,4-dichloro-phenylalanine, 2-chloro-penylalanine, 3,4-dihydroxy-phenylalanine, 3,5-dibromotyrosine, 3,3-diphenylalanine, 3-ethyl-phenylalanine, 3,4-difluoro-phenylalanine, 3-chloro-phenylalanine, 3-chloro-phenylalanine, 2-fluoro-phenylalanine, 4-amino-L-phenylalanine, homophenylalanine, 3-(8-hydroxyquinolin-3-yl)-1-alanine, 3-iodo-tyrosine, kynurenine, 3,4-dimethyl-phenylalanine, 2-methyl-phenylalanine, m-tyrosine, 2-naphthyl-alanine, 5-hydroxy-1-naphthalene, 6-hydroxy-2-naphthalene, meta-nitro-tyrosine, (beta)-beta-hydroxy-1-tyrosine, (beta)-3-chloro-beta-hydroxy-1-tyrosine, o-tyrosine, 4-benzoyl-phenylalanine, 3-(2-pyridyl)-alanine, 3-(3-pyridyl)-alanine, 3-(4-pyridyl)-alanine, 3-(2-quinolyl)-alanine, 3-(3-quinolyl)-alanine, 3-(4-quinolyl)-alanine, 3-(5-quinolyl)-alanine, 3-(6-quinolyl)-alanine, 3-(2-quinoxalyl)-alanine, styrylalanine, 4-iodo-phenylalanine, 4-nitro-phenylalanine, phosphotyrosine, 4-tert-butyl-phenylalanine, 2-(trifluoromethyl)-phenylalanine, 3-amino-L-tyrosine, 3,5-diiodotyrosine, 3-amino-6-hydroxy-tyrosine, tyrosine.

In some configurations of the invention, the aromatic amino acid is brominated or chlorinated on the aryl functionality, at any position of the aromatic ring.

In some configurations of the invention, the brominated or chlorinated aromatic amino acid is a brominated or chlorinated phenyl alanine (4Br or 4Cl).

In some configurations of the invention, in a peptide of structure (II), n is 1 or 2.

In some configurations of the invention, when n is 1 or 2, one or both of the amino acids is an aromatic amino acid; or one or both of the amino acids is a brominated amino acid.

In some configurations of the invention, the peptide is selected from:
DOPA-(AA)ₙ-O-M, wherein each of AA, n, O and M is as defined herein;
DOPA-(AA)ₙ-NH₂, DOPA-(AA)ₙ-NHR₁, DOPA-(AA)ₙ-NHR₁R₂+, or DOPA-(AA)ₙ-NR₁R₂R₃+, wherein each of R₁, R₂ and R₃, independently of the other is —H or a —C₁-C₅alkyl;
DOPA-Phe-Phe-OM, wherein M is as defined herein;
DOPA-Phe-Phe(4Br)-OM, wherein M is as defined herein;
DOPA-Phe(4Br)-Phe-OM, wherein M is as defined herein;
DOPA-Phe(4Br)-Phe(4Br)-OM, wherein M is as defined herein;
DOPA-Phe-OM, wherein M is as defined herein;
DOPA-Phe-Phe(4Cl)-OM, wherein M is as defined herein;
DOPA-Phe(4Cl)-Phe-OM, wherein M is as defined herein;
DOPA-Phe(4Cl)-Phe(4Br)-OM, wherein M is as defined herein;
DOPA-Phe(4Cl)-Phe(4Cl)-OM, wherein M is as defined herein;
DOPA-Phe(4Br)-Phe(4Cl)-OM, wherein M is as defined herein;
DOPA-Phe-OM, wherein M is as defined herein;
DOPA-Phe-NH₂;
DOPA-Phe(4Br)—NH₂;
DOPA-Phe(4Cl)—NH₂;
DOPA-Phe-Phe-OMe;
DOPA-Phe-Phe(4Br)—OMe;
DOPA-Phe-Phe(4Cl)—OMe;
DOPA-Phe(4Br)-Phe-OMe;
DOPA-Phe(4Br)-Phe(4Br)—OMe;
DOPA-Phe(4Cl)-Phe-OMe;
DOPA-Phe(4Cl)-Phe(4Br)—OMe;
DOPA-Phe(4Cl)-Phe(4Cl)—OMe;
DOPA-Phe(4Br)-Phe(4Br)—OMe;
DOPA-Phe-OMe; and
Cbz-DOPA-Phe-OH.

In some configurations of the invention, the formulation is in a form of a suspension, a dispersion or an emulsion comprising a liquid medium and the peptide.

In some configurations of the invention, the peptide is provided soluble in a liquid medium.

In some configurations of the invention, the formulation comprises a liquid medium and the peptide, the formulation being for forming antimicrobial films on a surface region, or for preventing attack or damage or degradation or decomposition or poisoning due to presence of a microbial infection source, or for application on live plants, on fruits and vegetables or seeds.

In some configurations of the invention, the formulation is an agricultural formulation for decreasing microbial load in a liquid medium or a surface.

In some configurations of the invention, the formulation is for
(i) decreasing microbial load by preventing microbial infection, propagation, attachment or spreading, by eradicating of microbial cells or virions in a target, after they have been established, or
(ii) repelling microbial settling or attachment or assembly on a surface of live plants, pre-harvested or post-harvested fruits, vegetables, flowers and seeds, or
(iii) improving growth, storage, handling, safety, effectiveness of live agricultural products, or
(iv) for preventing spoilage and production of microorganism-derived undesirable by-products.

In some configurations of the invention, the formulation is for preventing propagation or spreading of microorganism by prevention of assembly and production of new microbial cells.

In some configurations of the invention, the formulation is formulated as a disinfectant composition or as a preservative.

In some configurations of the invention, the formulation is in a form of a paint, a latex emulsion, a polymer emulsion, an adhesive, a sealant, a caulk, a mineral or pigment slurry, a printing ink, a pesticide formulation, a household product, a personal care product, a hygiene product, a metal working fluid, a pharmaceutical, a foodstuff, a food additive, or any packaging material.

In some configurations of the invention, the peptide is provided in a particulate form or an encapsulated form or present or held within a solid matrix.

In some configurations of the invention, the solid matrix is a porous matrix, a soluble matrix, a metallic matrix, or a polymeric matrix.

In some configurations of the invention, the formulation exhibits antibacterial, antifungal, antimycotic, antiparasitic, antiprotozoal, antiviral, antiinfectious, antiinfective and/or germicidal, algicidal, amoebicidal, microbicidal, bactericidal, fungicidal, parasiticidal, protozoacidal, or protozoicidal properties.

In some configurations of the invention, the formulation is an antiviral formulation.

In some configurations of the invention, the formulation is effective against bacteria from the genus Aeromonas, Arcobacter, Bacillus, Brochothrix, Campylobacter, Carnobacterium, Clostridium, Enterobacteriacae, Escherichia, Listeria, Pseudomonas, Salmonella, Serratia, Shigella, Staphylococcus, Vibrio, Yersinia, Erwinia, Pseudomonas pyocyanea, and Corynebacterium xerosis.

In some configurations of the invention, the formulation is effective against fungi selected from Aspergillus flavum and Penicillium chrysogenum; parasites, Balantidium, Cryptosporidium, Cyclospora, Giardia, Isospora, Microsporidia, Trichinella spiralis and Toxoplasma gondii; Fusarium oxysporum, Penicillium italicum, Colletotrichum gloeosporioides, Colletotrichum capsica, and Fusarium solani, Pythium, Pythium sp., and Sclerotium rolfsii.

In some configurations of the invention, the formulation is effective against viruses and infective virons of viruses selected from bacteriophages, coronaviridae/corona-virus, orthomyxoviridae, paramyxoviridae, Coxsackie family of viruses and adenoviridae family, Tobamovirus, and Tomato brown rugose fruit virus.

In some configurations of the invention, the formulation is effective against bacteriophages and viral infectors in human and non-human animals.

In some configurations of the invention, the formulation is an antiviral formulation wherein the viral infector is a human or a canine coronavirus.

Use of at least one antimicrobial peptide of structure (I) or (II), as defined herein, as an antimicrobial agent or as an antimicrobial agent for rendering antimicrobial properties to an object, to a formulation or to a combination of materials.

Use of at least one antimicrobial peptide of structure (I) or (II), as defined herein, in a method of eradicating or reducing microbial population in a solid or liquid medium.

Use of at least one antimicrobial peptide of structure (I) or (II), as defined herein, in a method of preparing an antimicrobial formulation, object or surface.

A film comprising a peptide of structure (I) or (II), as defined herein, or a film formed of a formulation comprising a peptide of structure (I) or (II).

Particles formed of a peptide of structure (I) or (II), as defined herein, wherein said particles are optionally in a form of capsules.

A method of eradicating or reducing a population of microorganism in a solid or liquid medium, the method comprises contacting or adding or treating or allowing interaction of said medium with a peptide of structure (I) or (II) as defined herein, or a formulation comprising same.

A method of inducing or endowing antimicrobial properties to a surface region of an object, the method comprises contacting said surface region with a peptide of structure (I) or (II) as defined herein or a formulation comprising same and optionally allowing said formulation to form a solid film of said peptide.

A method of reducing microbial load on or in an object, the method comprising contacting said object with a peptide of structure (I) or (II) as defined herein or a formulation comprising same and optionally allowing said formulation to form a solid film of said peptide A method or protecting live plants, post-harvest plants, fruits and vegetables, seeds, or seedings from attack by at least one microbial source or pathogen, the method comprising contacting same with a peptide of structure (I) or (II) as defined herein or a formulation comprising the peptide and optionally allowing said formulation to form a solid film of said peptide.

A method of protecting seeds against microbial attack by a microbial source or a pathogen, the method comprising contacting said seeds with a peptide of structure (I) or (II) as defined herein or a formulation comprising same and optionally allowing said formulation to form a solid film of said peptide.

A film or a coat formed of a formulation as defined herein.

In some configurations of the invention, the film is on a surface of an object.

In some configurations of the invention, the film is formed on an object selected from aesthetic objects, cosmetic objects, medical surfaces, consumer goods containers, means of transport, furnishing objects, common spaces, equipment, clothing, surfaces of medical devices, or a surface which favors proliferation of bacterial colonies and the adhesion of viral particles.

An antimicrobial formulation comprising at least one antimicrobial peptide of structure (I): DOPA-X, wherein X is an amino acid or a peptide comprising between 2 and 4 amino acids, wherein the peptide of structure (I) is free of F atoms or free of antifouling moieties or atoms, the formulation being in a form of an aerosol formulation, liquid formulation, spray formulation, dust formulation, dry flowable formulation, granulated formulation, wettable formulation, brushing formulation, or a polymeric formulation.

A solid matrix comprising an antimicrobial peptide of structure (I): DOPA-X, wherein X is an amino acid or a peptide comprising between 2 and 4 amino acids, wherein the peptide of structure (I) is free of F atoms or free of antifouling moieties or atoms.

In some configurations of the invention, the solid matrix is a porous solid material.

An antiviral liquid reagent comprising a peptide of structure (I) or (II) as defined herein, the reagent being for use as a disinfectant.

A disinfectant or a sterilizing agent comprising a liquid medium and a peptide of structure (I) or (II) as defined herein.

An antimicrobial formulation comprising an antimicrobial peptide consisting a peptide of structure (I) or (II) as defined herein.

An antiviral liquid formulation comprising at least one peptide of structure (II): DOPA-(AA)ₙ-M, wherein
DOPA is 3,4-dihydroxy-L-phenylalanin (DOPA), or a hydroxylated DOPA,
AA is an amino acid or an amino acid sequence or peptide comprising between 2 and 4 amino acids;
n is an integer between 1 and 4; and
M is a functionality or a group of atoms present at the terminal end of the peptide, M being:
   (1) —O-M, wherein —O— is an oxygen atom of the amino acid and M is selected from a metal cation (such as Li, Na, K, and other monovalent metal cations); —H; —C₁-C₅alkyl; —C₆-C₁₀aryl; —C(═O)—O—C₁-C₅alkyl; —C(═O)—O—C₆-C₁₀aryl; —C(═O)—O—C₁-C₅alkylene-C₆-C₁₀aryl; —NH₂; —NHR₁, —NHR₁R₂, —NR₁R₂R₃, wherein each of R₁, R₂ and R₃, independently of the other is —H or a —C₁-C₅alkyl; or
   (2) and amine or an ammonium selected from —NH₂; —NHR₁, —NHR₁R₂, —NR₁R₂R₃, wherein each of R₁, R₂ and R₃, independently of the other is —H or a —C₁-C₅alkyl.
wherein the peptide of structure (II) is free of F atoms or free of antifouling moieties or atoms.

In some configurations of the invention, the formulation comprises the peptide comprises two or three amino acids.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D present visual images of coatings (1 cm*1 cm) formed by the peptide DOPA-Phe(4F)-Phe(4F)—OMe. (a) 1 layer (b) 2 layers, (c) 3 layers, and peptide DOPA-Phe-Phe-OMe, and (d) 3 layers.

FIG. 2 provides a schematic illustration of the formation of a peptide-based antiviral coating and the peptide molecular structures.

FIGS. 3A-G provide characterization of the peptide-based coatings formed by drop-casting. SEM images of the assemblies formed by the peptide DOPA-Phe(4F)-Phe(4F)—OMe. (a) 1 layer (b) 2 layers, (c) 3 layers, and (d) a 3-layer coating of DOPA-Phe-Phe-OMe. (e) FT-IR analysis of the structures formed by DOPA-Phe(4F)-Phe(4F)—OMe (dashed line) and DOPA-Phe-Phe-OMe (black line). (f) Water contact angle measurements of bare glass and glass coated by DOPA-Phe(4F)-Phe(4F)—OMe coatings or DOPA-Phe-Phe-OMe. The analyses were carried out in triplicate. ANOVA and Duncan's test (SPSS, SPSS, Inc. Chicago, IL, USA, version 22) software were used to indicate the statistically significant differences among values (P<0.05). (g) X-ray photoelectron spectroscopy (XPS) analysis of bare glass and glass coated by DOPA-Phe(4F)-Phe(4F)—OMe or DOPA-Phe-Phe-OMe.

FIG. 4 provides ATR-FTIR spectrum for 3-layer coatings of DOPA-Phe(4F)-Phe(4F)—OMe and DOPA-Phe-Phe-OMe.

FIGS. 5A-H demonstrates the effect of the peptide coatings on the chemical inactivation of T4 bacteriophage and canine coronavirus (CCV). (a) The virus titers of T4 bacteriophage after incubation with bare glass, L-DOPA, diphenylalanine, DOPA-Phe-Phe-OMe (3 layers), DOPA-Phe(4Br)-Phe(4Br)—OMe (3 layers) for 24 h and UV irradiation for 3 h, (b) the log (TCID50/mL) of CCV after incubation with bare glass, L-DOPA, diphenylalanine, DOPA-Phe-Phe-OMe (3 layers), DOPA-Phe(4Br)-Phe(4Br)—OMe(3 layers) and UV irradiation for 3 h, (c-g) representative images of infected cells on bare glass, L-DOPA, diphenylalanine, DOPA-Phe(4Br)-Phe(4Br)—OMe, and DOPA-Phe-Phe-OMe, and (h) representative images of CRFK CCL94 cells in the medium.

FIG. 6 shows the virus titers of bacteriophage T4 treated on a bare glass, DOPA-Phe(4Br)-Phe(4Br)—OMe coating (1 layer), and a DOPA-Phe(4Br)-Phe(4Br)—OMe coating (2 layers).

FIG. 7 shows the log (TCID₅₀/mL) of CCV after incubation with different concentration of peptide solution.

FIGS. 8A-C provide representative images of (a) Untreated CRFK CCL94 cells in EMEM medium, (b) CRFK CCL94 cells treated by DOPA-Phe-Phe-OMe (10 mg/mL), and (c) CRFK CCL94 cells treated by DOPA-Phe(4Br)-Phe(4Br)—OMe (10 mg/mL).

FIGS. 9A-I provide characterization of the peptide assemblies in solution. (a-c) TEM, SEM, and AFM images for DOPA-Phe-NH2, (d-f) TEM, SEM, and AFM images for DOPA-Phe(4Br)—NH2, (g) DLS size distribution for DOPA-Phe-NH2 and DOPA-Phe(4F)—NH2, (h) CD spectra and (i) FTIR spectrum (Amide I) for DOPA-Phe-NH2 and DOPA-Phe(4Br)—NH2.

FIGS. 10A-E provide TEM images at different magnifications of (a, d) untreated T4 bacteriophage, (b, e) treated with DOPA-Phe-NH2, and (c, f) treated with DOPA-Phe(4Br)—NH2.

FIGS. 11A-G provide characterization of the peptide assemblies on the surface and their antiviral activity against bacteriophage T4. (a) Optical images of the coating formed by DOPA-Phe-NH2 (on the left) and DOPA-Phe(4Br)—NH2 (on the right), (b) ATR-FTIR spectrum for coatings formed by DOPA-Phe-NH2 or DOPA-Phe(4F)—NH2, (c-d) SEM and AFM images for coatings of DOPA-Phe-NH2 (e-f) SEM and AFM images or DOPA-Phe(4Br)—NH2, (g) the antiviral activity for peptide-based coatings against bacteriophage T4. “ND” represents “no virus was detected”.

FIGS. 12A-B demonstrates viability of HT-29 (a) and A2780 (b) cells based on the MTT assay.

### DETAILED DESCRIPTION OF EMBODIMENTS

### Materials and Methods

Peptides were purchased from GL Biochem (Shanghai) Ltd. with a purity >95%. L-DOPA, with a purity >98% was purchased from Tokyo Chemical Industry Co., Ltd. Diphenylalanine (H-Phe-Phe-OH), L-phenylalanine (L-Phe) was purchased from Bachem AG (Bubendorf, Switzerland) Co., Ltd. with a purity of 98%. Methanol, sodium dodecyl sulfate (SDS), and ethanol were purchased from Sigma Aldrich (St. Louis, Missouri, USA). Escherichia coli strain B (Migula) Castellani and Chalmers (ATCC 11303) and Escherichia coli bacteriophage T4 (ATCC 113030-B4) bacteria were purchased from the American Type Culture Collection (ATCC, Manassas, Virginia, USA). Agar and LB broth were purchased from Merck (New Jersey, USA) and Becton Dickinson (New Jersey, USA), respectively. The CRFK (ATCC® CCL-94™) cell line and Canine coronavirus (CCV) (ATCC® VR2068™) were purchased from Biological Industries, Beit-Haemek, Israel. Essential minimum Earl salts medium (EMEM), 200 mM L-Alanyl-L-Glutamine, Penicillin-Streptomycin-Amphotericin (PSA), Trypsin-EDTA, Dulbecco's Phosphate Buffered Saline (DPBS), and Donor Horse Serum (DHS) were purchased from Biological Industries, Beit-Haemek, Israel. Trypsin 1:250 was purchased from Bio-World, Dublin, OH, USA.

### Peptide Solution Preparation

The peptide stock solution was prepared by dissolving the lyophilized peptide powder in ethanol. Then, the stock solution was diluted by triple distilled water (TDW) and stirred for 3 h (150 rpm, room temperature). The final peptide concentration was 5.54 mM.

### Surface Modification

Prior to the coating procedure, glass surfaces (1 cm*1 cm) were washed for 30 min with each of three different solvents: 2% SDS, methanol, and Ethanol. Subsequently, the surfaces were dried by a flow of nitrogen and then left in UV-Ozone for 10 min. The single-layer coating was prepared by drop-casting 100 μL of peptide (10 mg/mL) solution on clean glass and then dried at room temperature. Subsequently, the double-layer coating was prepared by adding 100 μL peptide solution (10 mg/mL) on the dried 1-layer coating and finally dried at room temperature. The triple-layer coating was prepared by drop-casting three times as described before.

### Water Contact Angle Measurements

Contact angle measurements were examined by using a Theta Lite optical tensiometer (Attension, Finland). Each experimental measurement consisted of three repeats.

### Scanning Electron Microscopy (SEM)

The different dried surfaces were coated with gold using a Polaron SC7640 Sputter Coater. SEM images were taken using an extra high-resolution scanning electron microscope, Magellan TM400L, operating at 1 kV. The coverage degree of the surface by the structures was assessed using ImageJ. It was calculated using eq 1.

$$The coverage degree ( \% ) = \frac{The coverage area with spherical structures}{The total area of the SEM image}$$

### X-Ray Photoelectron Spectroscopy (XPS) Analysis

X-ray photoelectron spectroscopy (XPS) analyses were performed by using an AXIS Ultra X-ray photoelectron spectrometer (Kratos Analytical, Ltd., Manchester, UK). The sample's take-off angle was 90°. The vacuum pressure in the analyzing chamber was maintained at 2×10⁻⁹ Torr. High-resolution XPS spectra were collected for F is, O is, C is, and N is peaks with 20 eV pass energy and 0.1 eV step size. Data analyses were carried out using Kratos Vision data reducing processing software (Kratos Analytical, Ltd.) and Casa XPS (Casa Software, Ltd.).

### The Stability of Peptide Coatings

To examine the coating stabilities, peptide coatings were immersed into TDW for 5 min or washed with 2 mL TDW three times and then dried at room temperature. X-ray photoelectron spectroscopy (XPS) analyses were performed for the immersed and washed coatings. Moreover, peptide coatings were wiped with a finger at a load of 1 g and then water contact angle measurements were performed using the same method as described in the Contact angle measurements section.

### Fourier Transform Infrared Spectroscopy (FT-IR) and ATR-FTIR

FT-IR was recorded using a Nicolet 6700 FT-IR spectrometer with a deuterated triglycine sulfate (DTGS) detector (Thermo Fisher Scientific, MA, USA). Peptide solutions were deposited on a CaF₂ plate and dried by vacuum. The peptide deposits were resuspended with D₂O and subsequently dried, forming thin films. The resuspension procedure was repeated twice to ensure maximal hydrogen-to-deuterium exchange. The measurements were taken using a 4 cm⁻¹ resolution and averaged after 2000 scans. For ATR-FTIR analysis, ATR spectra were recorded using FT-IR (Thermo scientific, Model Nicolet 6700) with GeATR arrangement (Harrick Scientific's VariGATR). For all the surfaces spectra were 3 collected with an applied force of 350 N, at 4 cm⁻¹ resolutions with 3000 scans averaged signal and an incident angle of 65°.

### Antiviral Activity for Coatings Against T4 Bacteriophage (Bacteriophage Plaque Assay)

The antiviral activity performance was measured according to work by Kim et al. and Matsumoto et al. with some modification. The peptide coatings were prepared as described before. We used T4 bacteriophage as the virus when we measured the antiviral activity. Bacteriophage suspension was propagated on E. coli (ATCC 11303) according to ISO 18061, which was grown as the host strain. The mixture obtained by adding 100 μL of fresh T4 phage to an overnight culture of E. coli was incubated for 4 h in a 10 mL LB phage at 37° C. After multiplication, the virus was collected by centrifugation at 4000×g for 10 min at room temperature. The concentration of T4 bacteriophage was measured using the soft agar overlay (double-agar layer) plaque assay method. The supernatant containing the bacteriophage culture was filtrated through cellulose acetate filters (Millipore Acrodisc; the pore sizes were 0.45 μm and 0.22 μm) before use (the final concentration of bacteriophage was 10⁹ PFU/mL).

Ten decimal serial dilutions of the virus suspension were prepared by LB phage. The aqueous suspensions for the E. coli and T4 bacteriophage inactivation experiments contained sample surfaces having dimensions of 1*1 cm and T4 phage at 1.0×10⁶ plaque-forming units (PFU)/mL. As a positive control, we also added 16 μL 1.0×10⁶ plaque-forming units (PFU)/mL bacteriophage T4 on bare glass and then treated the surface by a UV irradiation (long wave ultraviolet 365 nm) for 3 h. Next, the phages were incubated under humid conditions at room temperature (25° C.) in a dark room for 24 h. After incubation, the phages were harvested by shaking with 2 mL SCDLP broth for 15 min to stop the incubation. The T4 in SCDLP bacteriophage was diluted with LB phage 10-fold. Subsequently, samples and bacteria were mixed with 0.6% agarose. Then the mixture was spread out on 1.5% LB agar to form a double agar layer. The plate was incubated at 37° C. for 18 h to form the plaques. The antiviral activity was defined and calculated as eq 2 below: The initial virus titer (No) and the virus titer after incubation (N) were calculated by counting the plaque number. For each sample, 9 repeats were performed to assess the antiviral activity.

$$Antiviral activity = {log}_{10} ( N / {N}_{0} )$$

### Antiviral Minimum Inhibitory Concentrations (MIC) in Solutions Against T4 Bacteriophage

All the tripeptide powders were firstly dissolved into ethanol and diluted by TDW. The solutions were stirring for 3 h (150 rpm, room temperature) for complete self-assembly. The final concentration is 10 mg/mL. Our previous study showed that the peptide could self-assemble into spheres. As a control, single amino acids (L-DOPA, L-Phenylalanine, and 4-Fluoro-L-phenylalanine) were also used to examine their antiviral activity. To further evaluate the antiviral activity for the peptide assemblies, the antiviral MIC for traditional Ag and Cu nanoparticles were also tested. The stock solutions were two-fold diluted using LB phage to obtain a series of peptide concentrations until the lowest concentration needed was achieved. Next, the 100 μL T4 bacteriophage (105 PFU/mL) and 100 μL diluted peptide solutions were transferred into 800 μL LB phage and then the samples were shaken at 150 rpm at room temperature. After 24 h incubation, the samples were centrifuged at 14000 g for 10 min to precipitate the peptide particles. Supernatants were collected and then 10-fold diluted once. Then, the 20 μL supernatants were mixed with 25 μL bacteria (E. coli. ATCC11303) in 1 mL warm 0.6% agarose. Then the mixture was spread out on 1.5% LB agar to form a double agar layer. The plate was incubated at 37° C. for 18 h to form the plaques. The lowest concentration of peptides that prevented virus growth is defined as the antiviral minimum inhibitory concentration (MIC).

### Antiviral Activity Against Canine Coronavirus (CCV)

Cell Culture: CRFK cells were propagated in a growth medium containing EMEM with 2 mM L-Alanyl-L-Glutamine, 1% PSA solution, and 10% DHS at 37° C. with 5% CO₂.

Virus propagation: CRFK cells were incubated in a 175 cm² flask until 80-90% confluent was achieved. After the flask was washed with 20 mL of DPBS, 2 mL of CCV solution were added and incubated at 37° C. with 5% CO₂. After a 2-hour incubation, EMEM (with 2 mM L-Glutamine and 1% PSA), DHS (final concentration 1%) and 1:250 Trypsin (final concentration 1 μg/mL) were added to the flask to a final volume of 20 mL. After the infected flask was incubated at 37° C. with 5% CO₂ for a week, the medium was centrifuged at 1000×g, at 4° C., for 10 min. Next, the supernatant was transferred to a sterile 20 mL Vivaspin tube with a 100,000 MWCO filter and then centrifuged at 2500 g, at 4° C. to concentrate the virus to a final volume of 1-2 mL and finally preserved in liquid nitrogen.

TCID50/mL Measurements: About 10,000 CRFK cells were incubated in each well using 96-well plates until 80-90% confluent was achieved. Then the examined CCV solution was diluted to a 10-fold dilution series. After the flask was washed with 0.2 mL of DPBS, 0.1 mL of the diluted CCV solution was added and incubated at 37° C. with 5% CO₂ (5-6 repeats for each dilution). After a 2-h incubation, EMEM (with 2 mM L-Glutamine and 1% PSA), DHS (final concentration 1%) and 1:250 Trypsin (final concentration 1 μg/mL) were added to the well to a final volume of 0.2 mL. The TCID₅₀/mL (tissue culture infectious dose at the 50% endpoint per mL) calculations were performed according to the improved Karber method.

Antiviral activity for peptide coatings: A 16 μL test viral suspension was inoculated onto the coated and uncoated glass surface and incubated for 3 h at room temperature. As a positive control, we added 16 μL viral suspension and incubated the substrate for 3 h under UV irradiation (longwave ultraviolet 365 nm). After the contact time, a 2 mL SCDLP broth was added to remove the viruses from the surface. From this mixture, a 10-fold dilution series was prepared and a TCID₅₀/mL measurement was carried out. Each experiment contained three coated surfaces and three uncoated surfaces, and three experiments were performed.

Antiviral activity for peptide solutions: A 20 μL viral suspension was inoculated into 180 μL peptide solution (10 mg/mL, 1 mg/mL, 0.1 mg/mL, 0.01 mg/mL, and 0 mg/mL) for 3 h at room temperature. From this mixture, a 10-fold dilution series was prepared and a TCID₅₀/mL measurement was carried out. Each sample at different concentrations was performed by three experiments.

### Results and Discussion

To self-assemble the peptides, we first dissolved the peptide in ethanol to a concentration of 100 mg/mL and then diluted them by triple distilled water to a concentration of 10 mg/mL. The peptide solutions were stirred for 3 h at 150 rpm. This process leads to the formation of spherical structures. To evaluate the antiviral activity of the assemblies, we measured the antiviral minimum inhibitory concentration (MIC) against T4 bacteriophage (see method section). The results are shown in Table 1. Peptide assemblies formed showed antiviral activity against T4 bacteriophage. The antiviral MIC were 62 g/mL and 31 g/mL. This indicated that the assemblies formed by the peptide have better antiviral activity against T4 bacteriophage. For comparison, we assessed the antiviral activity of metal nanoparticles and found that the MIC of silver nanoparticles and copper nanoparticles was 100 g/mL and 2500 g/mL (Table 1). This suggests that the peptide assemblies have better antiviral activity than those of traditional nanoparticles. We also determined the antiviral MIC against T4 bacteriophage for amino acids that comprises the peptides: L-DOPA, L-Phenylalanine, or 4-Fluoro-L-phenylalanine. The MIC values indicate that the individual amino acids have no antiviral activity against T4 bacteriophage (Table 1). These findings suggest that the antiviral activity can be attributed to the assemblies formed by the peptide and not by their components.

TABLE 1
Antiviral minimal inhibitory concentration (antiviral
MIC) against T4 bacteriophage for the tripeptide assemblies,
single amino acids and metal nanoparticles.

| | Antiviral MIC |
|---|---|
| Sample names | (μg/mL) |
| DOPA-Phe-Phe-OMe | 62 |
| DOPA-Phe(4F)-Phe(4F)-OMe | 31 |
| L-DOPA | — |
| L-Phe | — |
| L-Phe(4F) | — |
| AgNPs | 100 |
| CuNPs | 2500 |

“—” represents no antiviral MIC. All experiments are repeated for 3 times.

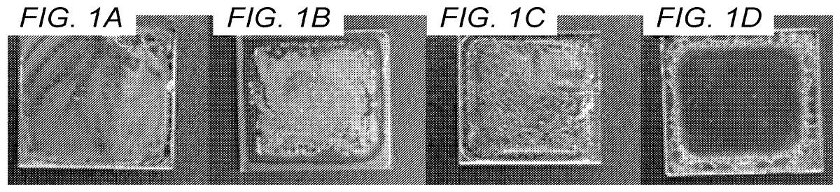

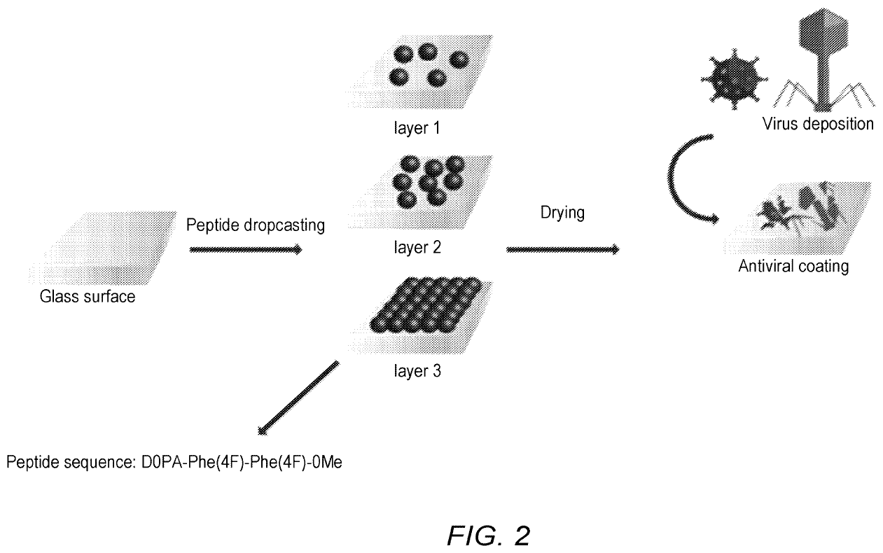

To generate a coating from these assemblies we simply drop-casted the peptide solution on a glass slide (FIG. 2). After drying the solution under ambient conditions, we observed a non-transparent film on the substrate (FIG. 1). This process was repeated to obtain 2 layers and once more to obtain 3 layers. The surfaces coated with 3 layers seemed more uniform and thicker than those with 2 layers (FIG. 1).

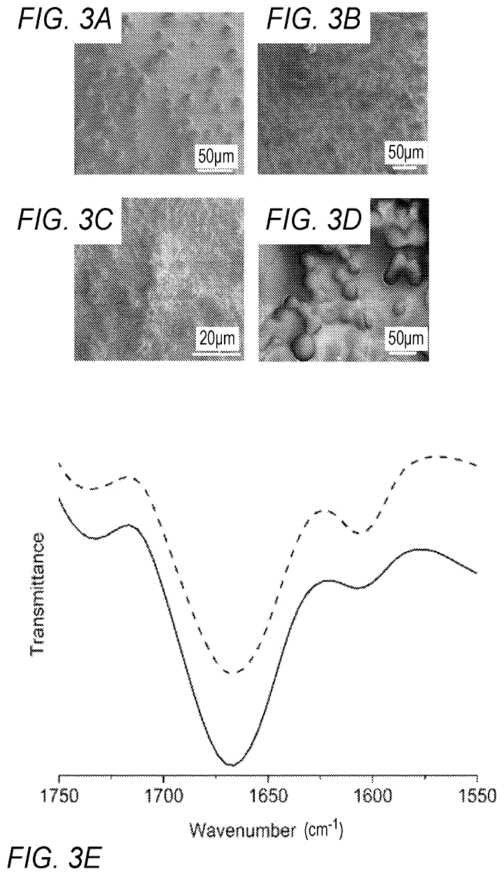

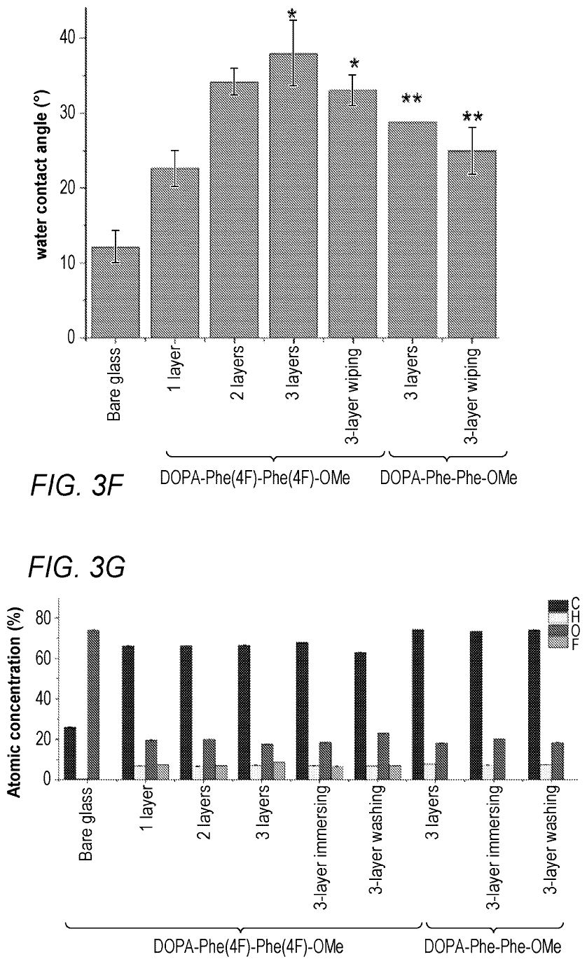

To characterize the morphology of the peptide-based coatings, we analyzed the surface using scanning electron microscopy (SEM). FIGS. 3A-C clearly shows the formation of spherical structures with a diameter of 8-10 μm on the surface. The surface coverage by the spherical structures increased when a second and a third layer were formed. When drop-casted only one time, the coating resulted in poor coverage, with only about 20% coverage area of the surface (based on ImageJ analysis, see the supporting information for more details). However, the surface coverage increased to nearly 80% when a second layer was applied and 95% for a 3-layer coating. Drop-casting the peptide solution of DOPA-Phe-Phe-OMe on a glass surface also resulted in spherical structures with a diameter of 10 μm (FIG. 3D).

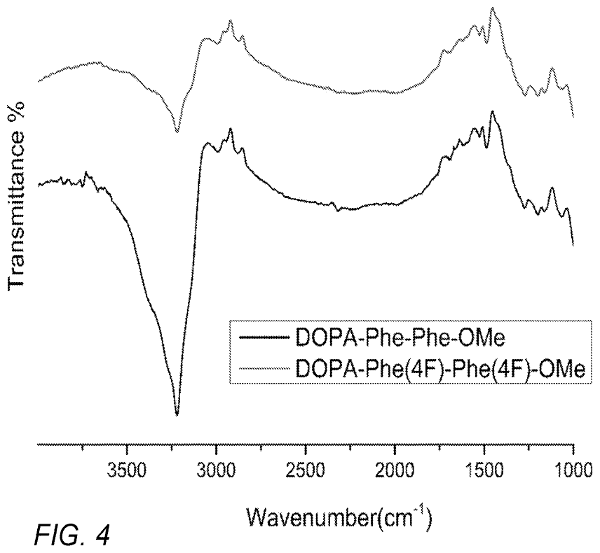

Next, we analyzed the structural properties of the spherical assemblies formed by the peptides using Fourier Transform Infrared spectroscopy (FT-IR). The spectrum for the assemblies formed in solution by the peptide DOPA-Phe(4F)-Phe(4F)—OMe had two distinctive peaks centered around 1606 cm⁻¹ and 1667 cm⁻¹ (FIG. 3E) that are correlated with β-sheet like structure. These results are in agreement with the structural analysis we previously performed for the assembles formed by this peptide. The IR spectrum for DOPA-Phe-Phe-OMe had similar peaks at 1607 cm⁻¹ and 1667 cm⁻¹. To evaluate the self-assembly structure on the glass substrates, ATR-FTIR was used. The ATR-FTIR spectrum around 1800-1500 cm⁻¹ is related to the stretching band of amide I and can indicate the secondary structure of the peptides. From FIG. 4, DOPA-Phe-Phe-OMe and DOPA-Phe(4F)-Phe(4F)—OMe coated substrates comprise two peaks at 1618 cm⁻¹ and 1622 cm⁻¹, respectively, indicating an antiparallel beta-sheet.

To determine the hydrophobicity of the coatings, we measured the water contact angle of the coated surfaces (FIG. 3F). We noticed an increase in the water contact angle for all coated surfaces when compared with that of bare glass (12±2°). This implies that the peptide was deposited on the surface. Coated surfaces with DOPA-Phe(4F)-Phe(4F)—OMe had a water contact angle of 23±2° for 1 layer, 34±2° for 2 layers and 38±4° for 3 layers. This increase in hydrophobicity is in accordance with the surface coverage of the peptide; when the surface coverage increased, the hydrophobicity of the surface increased as well (FIGS. 3A-C and FIG. 1). The water contact angle of the glass also increased with DOPA-Phe-Phe-OMe to 29±1° (peptide was drop-casted 3 times). As expected, this value is lower than the value for the fluorinated peptide.

To ensure the modification of the glass surfaces by the peptides, we measured the X-ray photoelectron spectroscopy (XPS). The atomic concentration of carbon, fluorine, and nitrogen increased upon increasing the number of DOPA-Phe(4F)-Phe(4F)—OMe peptide layers (FIG. 3G). With DOPA-Phe-Phe-OMe, the atomic concentration of carbon and nitrogen also increased when compared with bare glass. These findings indicate that the peptides are indeed deposited on the glass surface.

To investigate the stability of the coating on the surface, we performed XPS analysis for a 3-layer coating for both peptides, after immersing the coating in a water bath containing water (TDW) for 5 min or washing it three times using a pipette filled with TDW. Importantly, the intensity of the fluorine, carbon, and nitrogen signals did not decrease after this treatment (FIG. 3G). This indicates that the peptide coating adhered well to the glass and was not harmed by dipping and washing the surface. The same trend was observed for the peptide DOPA-Phe-Phe-OMe: both the carbon and nitrogen signals did not change appreciably after dipping and washing the surface. Furthermore, we examined the water contact angle of the coated surfaces after wiping them with a wet paper towel at a load of 1 g. The water contact angle for a 3-layer coating of DOPA-Phe(4F)-Phe(4F)—OMe and DOPA-Phe-Phe-OMe (after wiping) was 33±1° and 25±2°, respectively (FIG. 3F). This wiping force did not appreciably affect the water contact angle of the 3-layer coatings (P>0.05). This indicates that the external physical force did not harm the coatings.

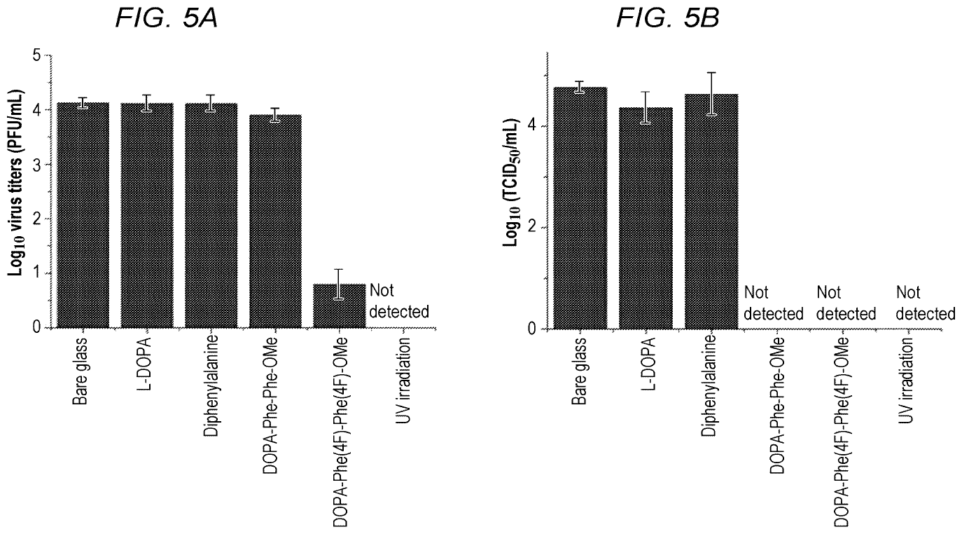

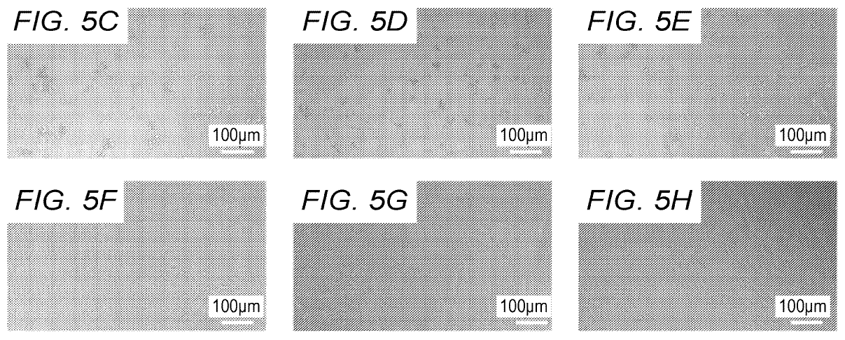

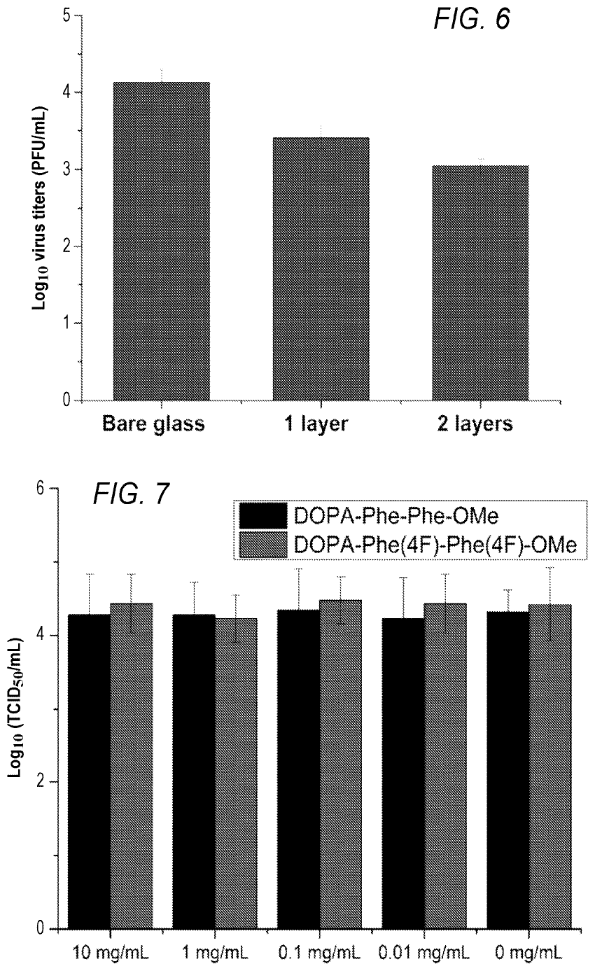

To determine the antiviral activity of the peptide-based coating, we incubated about 10⁶ T4 bacteriophages on each surface. T4 bacteriophage is a DNA-based virus that infects E. coli and causes them to burst. FIG. 5A and FIG. 6 present the results of the inactivation of T4 bacteriophage for each sample. As shown, the virus titers for the glass-coated surfaces substantially decreased when compared to bare glass (13579±2839 PFU/mL): 8029±205 PFU/mL for a 3-layer coating of DOPA-Phe-Phe-OMe and 3±6 PFU/mL for a 3-layer coating of DOPA-Phe(4F)-Phe(4F)—OMe, respectively. We also noted that the reduction rates (log (N/No)) of virus titers for the DOPA-Phe-Phe-OMe coating (3 layers) and the DOPA-Phe(4F)-Phe(4F)—OMe coatings (1 layer, 2 layers, and 3 layers) were −0.2, −0.7, −1.1, and −3.7, respectively. It can be inferred that both DOPA-Phe-Phe-OMe and DOPA-Phe(4F)-Phe(4F)—OMe reduced the virus titers and exhibited antiviral activity against T4 bacteriophage. The antiviral activity of the coating formed by DOPA-Phe(4F)-Phe(4F)—OMe increased with surface coverage. Not only 3-layer coating has antiviral activity but also 1 layer and 2 layers present good antiviral activity. The viral titer of 1 layer and 2 layers are 2610±369 PFU/mL and 1111±201 PFU/mL respectively. When compared to bare glass, the reduction of viral titer when compared with that of bare glass in percentage is 79% and 91% for 1 layer and 2 layers, respectively. A 3 layer coating has the best activity as it reduces the viral titer by 99.9% when compared to bare glass. SEM analysis of the coated surfaces (FIGS. 3A-C) shows that the surface coverage by a 3-layer coating was higher than 1-layer or 2-layer coatings. The additional peptide assemblies using a 3-layers coating provided more interactions between the peptide assemblies and bacteriophage T4 and therefore better antiviral activity. The 3-layer coating reduced the virus titers by 3 log, when compared to the glass surfaces and by 6 log when compared to the stock solution. Interestingly, the virus titers on a 3-layer coating of DOPA-Phe-Phe-OMe also decreased when compared with glass. This coating reduced the viral titers by 0.2 log. These results indicate that fluorinated phenylalanine plays a vital role in enhancing antiviral activity. To better understand which component endows the peptide with its antiviral activity against T4 bacteriophage, we examined the antiviral activity of L-DOPA and diphenylalanine (H-Phe-Phe-OH) by drop-casting each of them three times on glass surfaces. The results indicated that both L-DOPA and diphenylalanine did not decrease the viral titers when compared to a glass surface (FIG. 5A). For the positive control exposed to UV irradiation, no bacteriophage T4 could be detected.

Based on these results, we can conclude the DOPA is not essential for antiviral activity; however, it is necessary when the peptide interacts with a glass surface. Several studies showed that phenylalanine and its derivatives have antiviral and antifungal activity. Glycyrrhizic acid conjugates with phenylalanine and has antiviral activity against H1N1. Benzenesulfonamide-containing phenylalanine derivatives inhibit HIV-1 capsid formation. In addition, fluorinated compounds are known as antiviral agents. This includes 3′-fluoropenciclovir analogues, polyfluoroflavones, perfluoroalkyl derivatives of teicoplanin and vancomycin, 6′-fluorinated-aristeromycin analogues, fluorinated TiO₂, and other fluorinated molecules. These observations can explain our results with both DOPA-Phe-Phe and DOPA-Phe(4F)-Phe(4F).

It is important to note that the assemblies formed by Phe-Phe (the assemblies were prepared according to our previous study) and deposited on the glass did not exhibit antiviral activity. We assume that this is due to the lack of DOPA in the sequence and consequently, the instability of the peptide assemblies on the surface.

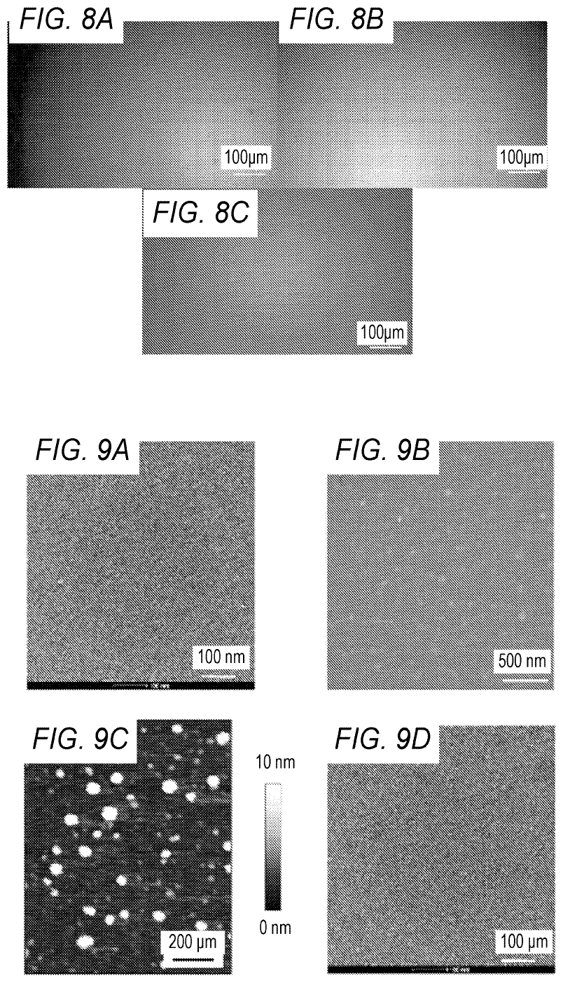

To examine the antiviral activity of the peptide with coronavirus, we used a corona surrogate—canine coronavirus (CCV). This is an enveloped, positive-stranded RNA virus with specific sequence homology with SARS-CoV-2 (36.93% sequence homology to SARS-CoV-2 of spike protein). Briefly, we inoculated CCV with a concentration (log (TCID₅₀/mL)) of 6.79±0.17 on uncoated and coated glass surfaces for 3 hours. The log (TCID₅₀/mL) (FIG. 5B) and the microphotographs of infected cells were assessed after incubation for one week (FIGS. 5C-G). Interestingly, the log (TCID₅₀/mL) for the 3-layer coating of DOPA-Phe-Phe-OMe and DOPA-Phe(4F)-Phe(4F)—OMe decreased below the detection limit of the system, whereas the log (TCID₅₀/mL) of uncoated coatings with CCV was 4.77±0.12. This means that both coatings reduced the viral load by over <99% when compared to bare glass. Importantly, the log (TCID₅₀/mL) of 3-layer coatings formed by either L-DOPA or diphenylalanine was 4.37±0.31 and 4.67±0.42, respectively. This value is similar to the log (TCID₅₀/mL) of bare glass (4.77±0.12). It implies that L-DOPA and diphenylalanine do not appreciably affect the viability of CCV. Meanwhile, the positive control (UV irradiation) also showed that no virus was detected. Moreover, we examined the antiviral activity of the peptide solution (FIG. 7). Interestingly, The log (TCID₅₀/mL) of CCV treated by both tripeptides for 3 h did not decrease compared with that of untreated CCV (4.32±0.45). These results indicate that the peptide solution is not antiviral while the peptide assemblies on the surface have antiviral activity. This data suggest that the antiviral activity of the peptide results from the peptide aggregation. This suggestion is supported by a recent publication that demonstrated that aggregates of phenylalanine act as membrane-disrupting molecules. We also examined the cell morphology when they were exposed to the peptide assemblies (FIG. 8). It can be clearly seen that the peptide assemblies (10 mg/mL) did not affect the cell morphology when compared with untreated host cells. Interestingly, the peptide assemblies in solutions only showed antiviral activity against bacteriophage T4 rather than CCV. We speculate that this difference originates from different capsid proteins arrangement in T4 phage versus CCV. The detailed antiviral mechanism will be carried in future work. It should be noted that DOPA, L-Phe, and diphenylalanine did not show any antiviral activity against both viruses indicating that the spherical supramolecular structures of peptide assemblies contributed to antiviral activity.

### Ultrashort Peptides

Two dipeptides, DOPA-Phe-NH₂ and DOPA-Phe(4F)—NH₂, were examined for their antiviral activity in solution and as a coating. The two peptides comprise one 3,4-dihydroxy-L-phenylalanine (DOPA) and one phenylalanine or fluorinated phenylalanine. L-DOPA is the main constituent of mussel adhesive proteins (MAPs) that can adhere to almost any substrate and can function under harsh conditions. Phenylalanine and fluorinated phenylalanine are aromatic residues that can mediate peptide self-assembly through π-π stacking.

To investigate the antiviral activity of DOPA-Phe-NH₂ and DOPA-Phe(4F)—NH₂ in solution, we performed an antiviral assay to identify the minimal inhibitory concentration (MIC) of each peptide. We first dissolved the peptides in ethanol to a concentration of 100 mg/mL and then diluted the solutions with triple distilled water to several concentrations. Both DOPA-Phe-NH2 and DOPA-Phe(4F)—NH2 exhibited antiviral activity against bacteriophage T4. The antiviral MIC was 125 g/mL and 62.5 μg/mL for DOPA-Phe-NH2 and DOPA-Phe(4F)—NH2, respectively, indicating that DOPA-Phe(4F)—NH2 had better antiviral activity than DOPA-Phe-NH2. This suggests that fluorinated residues can enhance the antiviral activity of the peptide. It follows our previous report on self-assembled tripeptides that have a similar antiviral activity with antiviral MIC of tens of g/mL.25 The importance of phenylalanine and fluorinated phenylalanine in antiviral peptides has been reported before. The improvement in antiviral activity upon the introduction of fluorine atoms is also in accordance with several previous reports that show that incorporating one or several fluorine atoms into an organic molecule can improve the pharmacokinetic and pharmacodynamic properties such as absorption, tissue distribution, secretion, the route and rate of biotransformation, toxicology, bioavailability, metabolic stability, and lipophilicity. Specifically, it was demonstrated that the peptide carbobenzoxy-D-phenylalanine-L-phenylalanine-glycine acts as an inhibitor of membrane fusion. In addition, it was shown that the fluoro-group at the phenyl ring in a triazole-dipeptide hybrid is essential for the antiviral peptide activity. To explore the antiviral mechanism for the dipeptide assemblies, we also evaluated the antiviral activity against bacteriophage T4 for L-DOPA, L-phenylalanine(L-Phe), fluorinated phenylalanine (L-Phe(4F)), diphenylalanine (Phe-Phe), and fluorinated diphenylalanine (Phe(4F)-Phe(4F)—OMe). As shown in Table 2, a single amino acid and the dipeptides Phe-Phe and Phe(4F)-Phe(4F)—OMe did not decrease the viral titer. We included copper nanoparticles (CuNPs) as a positive control in the antiviral MIC experiment. The antiviral MIC for CuNPs is 2500 g/mL much higher than the peptides MIC.

TABLE 2
Antiviral MIC for L-amino acids and dipeptides.

| | Antiviral MIC |
|---|---|
| Peptide sequences | (μg/mL) |
| L-DOPA | — |
| L-Phe | — |
| L-Phe(4F) | — |
| DOPA-Phe-NH2 | 125.00 |
| DOPA-Phe(4F)-NH2 | 62.50 |
| Diphenylalanine | — |
| Phe(4F)-Phe(4F)-Ome | — |
| CuNPs | 2500 |

“—” represents no antiviral activity

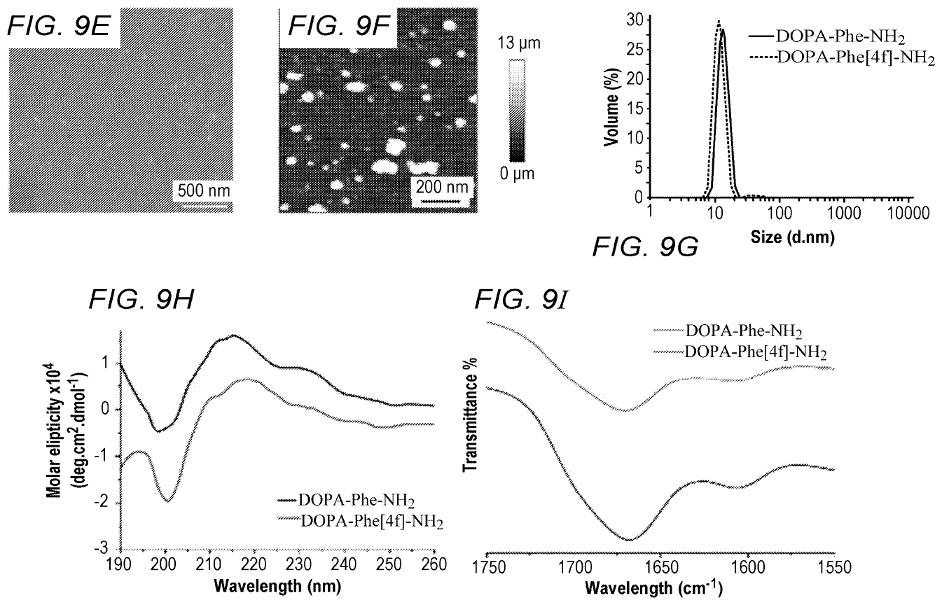

To investigate in what form the peptides exist in the solution at the MIC concentration, a transmission electron microscope (TEM) was used. The TEM analysis for the dipeptide DOPA-Phe-NH2 revealed that the peptide self-assembles into spherical nanoparticles with a size ranging from several nanometers up to 20 nm (FIG. 9A). Using scanning electron microscopy (SEM) and atomic force microscopy (AFM) (FIG. 9B-C) we were able to detect spherical structures with a diameter of 10-50 nm. Interestingly, there was no appreciable effect on the particle size when comparing DOPA-Phe-NH2 and DOPA-Phe(4F)—NH2, indicating that fluorine modification on the benzene ring of the Phe residue did not influence the peptide self-assembly (FIG. 9D-F). The slight difference in size between TEM and SEM may result from the sample preparation procedure where for TEM the sample is dried on a grid and negatively stained using uranyl acetate. Dynamic light scattering (DLS) analysis which is performed in solution revealed that the size of the self-assembled structures ranges from 10 to 50 nm. (FIG. 9G). Circular Dichroism (CD) and Fourier-transform infrared (FT-IR) spectroscopy were used to determine the secondary structure of the peptides (FIG. 9H-I). The CD spectra of both peptides exhibited a negative peak at approximately 200 nm and a positive peak at approximately 215 nm. This result suggests a 3-turn structure for the peptides assemblies. In addition, the FT-IR spectra for DOPA-Phe-NH2 and DOPA-Phe(4F)—NH2 showed a distinct peak at around 1668 cm-1 and 1670 cm-1, respectively, implying a β-turn structure.30 The CD and FT-IR spectrum of the two dipeptides were similar suggesting that introducing fluorinated atoms into the peptide sequence did not change the structure of the assemblies. The ordered structures could be formed by the π-π stacking of aromatic amino acids, hydrogen bonds, and electrostatic repulsion of —NH2 at the N-termini and C-termini.

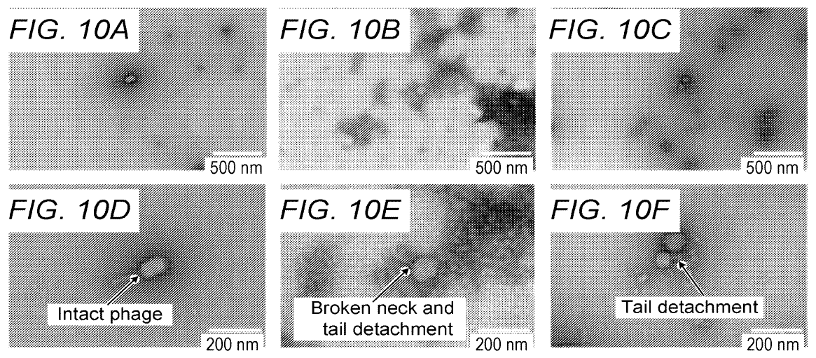

To better understand the mechanism of antiviral activity, we incubated a solution of bacteriophage T4 with the peptide assemblies at the peptide MIC concentration for 24 h and performed a TEM analysis (FIG. 10B-F). As a control, a solution of bacteriophage T4 without any peptide assemblies was analyzed (FIGS. 10A and 10D). In this control sample, the bacteriophage exhibited a typical structure of the viral head, tail, and long tail fibers. In contrast, when the bacteriophage was exposed and incubated with the peptide assemblies, the tail was detached from the head (FIG. 10B-F). This detachment was detected with both types of peptides assemblies (marked with an arrow). The head of bacteriophage T4 is attached to the tail via the neck proteins gp13 and gp14; subsequently, six 500 Å long, trimeric ‘whisker’ fibers (gpWac) are attached to the neck. Based on this information, we suggest that by interacting with the neck proteins, the peptide assemblies can disturb the protein structure. Moreover, significant damage to the morphology of the head could also be observed (FIG. 10B-F); the head did not exhibit an elongated icosahedron compared with the bacteriophages that were not exposed to the assemblies. We suggest that the damage to the morphology of the head could be due to the interaction and disruption between the bacteriophage capsid and the peptide assemblies. Importantly, the long tail fibers were also separated from the T4 tail. It has been reported that the long tail fiber consists of four proteins (gp34, gp35, gp36, and gp37) that recognize the receptor-binding site on the host cell. The destruction of the long tail fibers could lead to unrecognition for E. coli 11303 and could cause deactivation. As a positive control, we also examined the morphology of bacteriophages exposed to copper NPs (CuNPs) at an antiviral MIC. TEM analysis indicates that the CuNPs can also destroy the viral structures.

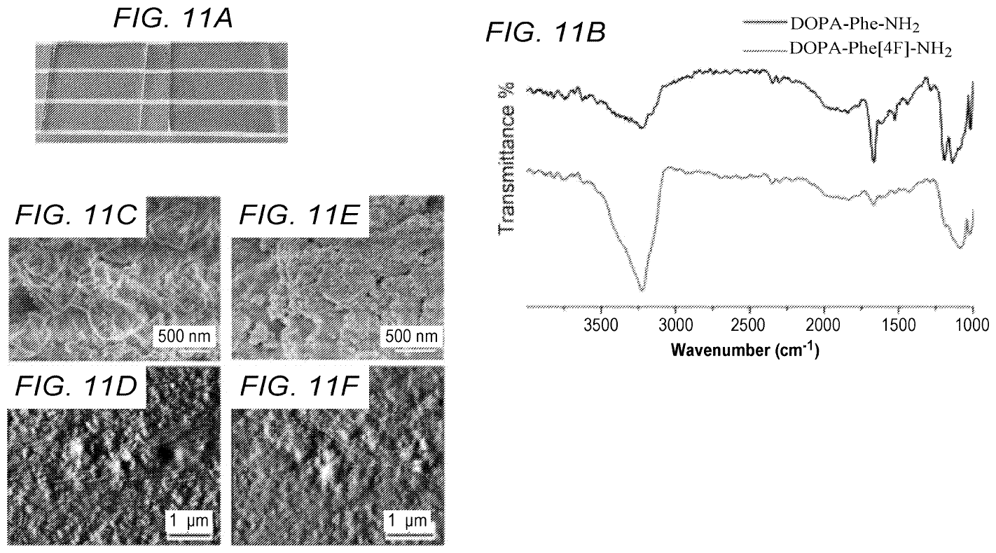

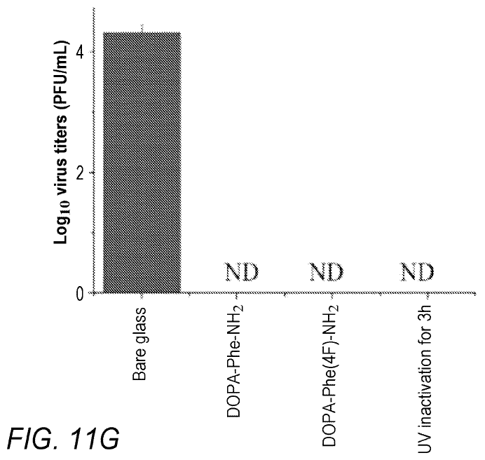

To further utilize the self-assembled particles to generate an antiviral coating we used a protocol we reported before. A peptide solution of DOPA-Phe-NH2 or DOPA-Phe(4F)—NH2, at a concentration of 10 mg/mL, was drop-casted three times on a clean glass substrate. This process resulted in transparent surfaces (FIG. 11A). SEM analysis reveals aggregates of spherical nanoparticles formed by DOPA-Phe-NH2 on the surface. (FIG. 11C-D). This aggregation is probably due to the high concentration of assemblies and the drying process. AFM topography images show that the aggregates are not uniform (FIG. 11D). Similar aggregated structures were obtained by DOPA-Phe(4F)—NH2 (FIG. 11E-F). The ATR spectra of the assemblies formed on the surface by the peptides are shown in FIG. 11B. The IR region 1800-1500 cm-1 is associated with the stretching band of amide I and indicates the secondary structure of the peptides. The Attenuated total reflection Fourier-transform infrared (ATR-FTIR) spectrum of a glass substrate coated with DOPA-Phe-NH2 had one main peak at 1666 cm-1, indicating a β-type structure.34,35 For the DOPA-Phe(4F)—NH2 coating, a similar peak appeared at 1669 cm-1; this suggests that both peptides form similar assemblies on the surface. The other regions of the spectrum had a low signal-to-noise ratio and significant peaks could not be detected. This is probably due to the tendency of this peptide to form spherical aggregates rather than a homogenous coating on the substrate.

We investigated the antiviral activity of the peptide-based coatings against bacteriophage T4. A solution of bacteriophages at a concentration of 1.0×106 plaque-forming units (PFU)/mL was applied to the peptide-based coating and incubated for 24 h. Then, the virus titer was determined by counting the number of plaques. As shown in FIG. 11G, no viruses were detected for both the coatings formed by either DOPA-Phe-NH2 or DOPA-Phe(4F)—NH2. On a bare glass, the viral titer was 29000±2000 PFU/mL and from that, we can deduce that the peptide-based coatings can reduce the viral titer by nearly 4 logs. This suggests that both peptide coatings can deactivate more than 99.9% of the viruses. The surface with T4 bacteriophage was treated by UV inactivation for 3 h as positive control and the bare glass with ethanol washing as a negative control to confirm that the antiviral activity is from the peptide itself. The virus titers treated by UV light are below the limit of detection.

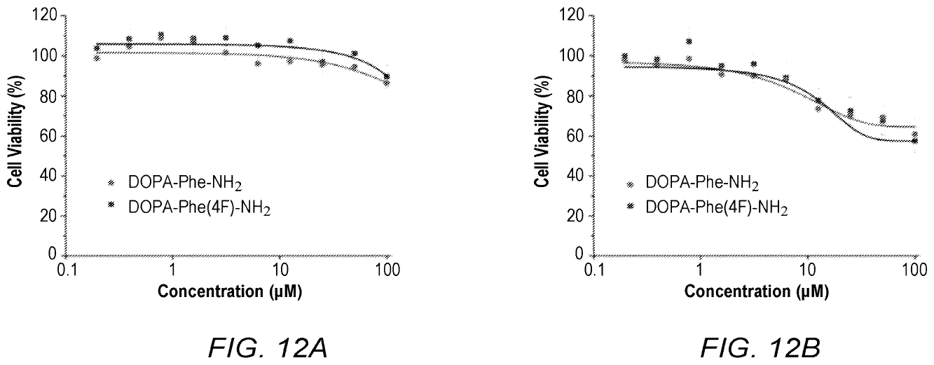

The cytotoxicity of the peptides was measured toward both colorectal adenocarcinoma (HT-29) and ovarian carcinoma (A2780) cancer cell lines. The cytotoxicity was tested by using the MTT (3-(4,5-dimethylthiazolyl)-2,5-diphenyltetrazolium bromide) assay after incubation of the cells with the peptides for 24 h. Both peptides showed very low cytotoxicity toward the HT-29 cells (FIG. 12A). The viability of the more sensitive cells, A2780, was slightly lower for both peptides and reached a plateau at around 60% at high concentrations. (FIG. 12B). These results suggest that both peptides have low cytotoxicity toward the cells.

### Experimental

### Materials

Phe(4F)-Phe(4F)—OMe, DOPA-Phe-NH2, and DOPA-Phe(4F)—NH2 were purchased from GL Biochem (Shanghai) Ltd. with a purity>95%. L-DOPA with a purity >98% was purchased from Tokyo Chemical Industry Co., Ltd. L-Phenylalanine, L-Phe(4F), and Diphenylalanine (H-Phe-Phe-OH) were purchased from Bachem AG (Bubendorf, Switzerland) Co., Ltd. with a purity of 98%. Methanol, sodium dodecyl sulfate (SDS), ethanol, Roswell Park Memorial Institute (RPMI) 1640 medium, 3-(4,5-dimethylthiazolyl)-2,5-diphenyltetrazolium bromide (MTT) and Isopropanol were obtained from Sigma Aldrich. were purchased from Sigma Aldrich (St. Louis, Missouri, USA). Escherichia coli strain B (Migula) Castellani and Chalmers (ATCC 11303) and Escherichia coli bacteriophage T4 (ATCC 113030-B4) bacteria were purchased from the American Type Culture Collection (ATCC, Manassas, Virginia, USA). Agar and LB broth were purchased from Merck (New Jersey, USA) and Becton Dickinson (New Jersey, USA), respectively. Ovarian carcinoma A2780 was purchased from European Collection of Authenticated Cell Cultures and colorectal adenocarcinoma HT-29 was purchased from American Type Culture Collection. Fetal Bovine Serum, 1% Penicillin-Streptomycin, and 1% L-Glutamine, were purchased from Biological Industries (Beit Haemek, Israel).

Preparation of the Peptide Assemblies and Peptide Coatings The dipeptide assemblies were prepared by dissolving the peptide powder in ethanol at 100 mg/mL; then they were diluted using triple distilled water (TDW) according to our previous work.16 The peptide coatings were prepared by drop-casting 3 times with 10 mg/mL peptide assemblies.

### Antiviral Minimal Inhibition Concentration (MIC) Against Bacteriophage T4

A series of two-fold diluted peptide solutions (1000, 500, 250, 125, 62.5, 31.25, 15.625, and 7.8125 g/mL) was prepared to measure the antiviral MIC. Next, 100 μL T4 bacteriophage (105 PFU/mL) and 100 μL diluted peptide solutions were transferred into 800 μL LB phage; then, the samples were shaken at 150 rpm at room temperature. After 24 h incubation, the samples were centrifuged at 14000×g for 10 min to precipitate the peptide particles. Supernatants were collected and then 10-fold diluted once. Next, the 20 μL supernatants were mixed with 25 μL bacteria (E. coli. ATCC11303) in 1 mL of warm 0.6% agarose. Then, the mixture was spread out on 1.5% LB agar to form a double agar layer. The plate was incubated at 37° C. for 18 h to form plaques. The lowest concentration of peptides that prevented virus growth is defined as the antiviral minimum inhibitory concentration (MIC). L-DOPA, L-phenylalanine, L-Phe(4F), Phe(4F)-Phe(4F), and diphenylalanine were used as controls.

### Transmission Electron Microscopy (TEM) for the Peptide Assemblies.

The peptide assemblies at antiviral MIC (125 g/mL and 62.5 g/mL for DOPA-Phe-NH2 and DOPA-Phe(4F)—NH2, respectively) were characterized by using Tecnai 12 TEM 120 kV (Phillips, Eindhoven, the Netherlands). Firstly, a carbon Formvar-coated copper grid was placed on a drop of peptide solution. Then, the samples were negatively stained by adding 5 μL of 2% uranyl acetate for 40 sec and dried in room temperature. The samples were transferred to TEM characterization immediately after preparation.

### Scanning Electron Microscopy (SEM) for the Peptide Assemblies and Coating

SEM measurements were performed by an extra high-resolution scanning electron microscope, Magellan TM400L, operating at 1 kV. A solution of peptide assemblies at antiviral MIC (125 g/mL and 62.5 g/mL for DOPA-Phe-NH2 and DOPA-Phe(4F)—NH2, respectively) was drop casted on glass surface and allowed to dry at room temperature. Then, the peptide assemblies and coatings were coated with gold using a Polaron SC7640 sputter coater and then observed.

### Atomic Force Microscope (AFM) for the Peptide Assemblies and Coating

The peptide assemblies were prepared at antiviral MIC (125 g/mL and 62.5 g/mL for DOPA-Phe-NH2 and DOPA-Phe(4F)—NH2, respectively) and then were drop-casted on the clean glass substrate. All AFM images of the peptide assemblies and coatings were taken in AC mode with a Si3N2 tip with a spring constant of 3 N/m using JPK NanoWizard®.

### Dynamic Laser Scattering (DLS)

A Malvern dynamic laser scattering (DLS) instrument (Zetasizer Nano ZSZEN3600) was used to determine the size distribution of the peptide assemblies. The size distribution of peptide assemblies at antiviral MIC (125 g/mL and 62.5 g/mL for DOPA-Phe-NH2 and DOPA-Phe(4F)—NH2, respectively) was performed.

### Circular Dichroism (CD)

The CD spectra were collected in a J-810 spectropolarimeter (JASCO, Tokyo, Japan), using a 0.1 cm pathlength quartz cuvette for far-UV CD spectroscopy (in the spectral range between 190 and 260 nm with a step width of 0.05 nm) at 20° C. The peptides were dissolved in TDW (0.1 mg/ml) and then filtered by using a 0.22 μm filter. The spectra for each sample spectra were collected three times, averaged, and the background (TDW) was subtracted.

### Fourier Transform Infrared Spectroscopy (FT-IR)

FT-IR was recorded using a Nicolet 6700 FT-IR spectrometer with a deuterated triglycine sulfate (DTGS) detector (Thermo Fisher Scientific, MA, USA) at a 4 cm-1 resolution and averaged after 2000 scans. Peptide solutions were deposited on a CaF2 plate and dried by vacuum. The peptide deposits were resuspended with D2O and subsequently dried, forming thin films. The resuspension procedure was repeated twice to ensure maximal hydrogen-to-deuterium exchange.

### Transmission Electron Microscopes (TEM) for T4 Bacteriophage

The phage stock solution at a concentration of 1·109 PFU/ml was diluted to 6-107 PFU/ml in DDW and added to either the peptide solution or TDW. The final concentration of the peptides DOPA-Phe-NH2 and DOPA-Phe(4F)—NH2 were 25 mg/ml and 12.5 mg/ml, respectively. The solutions were incubated for 24 h at 37° C., 120 rpm.

Then, a 10 μL drop of each sample was added to the grid for 30 sec and the excess was blotted with filter paper. The samples were negatively stained by adding 5 μL of 2% uranyl acetate for 40 sec and the excess was blotted with filter paper.

The samples were analyzed by using Tecnai 12 TEM 120 kV (Phillips, Eindhoven, the Netherlands) equipped with Phurona camera and RADIUS software (Emsis GmbH, Münster, Germany).

### Attenuated Total Reflection Fourier-Transform Infrared (ATR-FTIR)

ATR-FTIR spectra were collected with an applied force of 350 N, at 4 cm-1 resolution with 3000 scans averaged signal and an incident angle of 65°.

### Antiviral Activity Against Bacteriophage T4 for Peptide Coatings

The antiviral activity performance was measured according to our previous work. Briefly, 10 decimal serial dilutions of the virus suspension were prepared by LB phage. The aqueous suspensions for the E. coli and T4 bacteriophage inactivation experiments contained sample surfaces with dimensions of 1*1 cm and T4 phage at 1.0×106 plaque-forming units (PFU)/mL. Next, the phages were incubated under humid conditions at room temperature (25° C.) in a dark room for 24 h. After incubation, the phages were harvested by shaking them with 2 mL SCDLP broth for 15 min to stop the incubation. The T4 in the SCDLP bacteriophage was diluted with LB phage 10-fold. Subsequently, samples and bacteria were mixed with 0.6% agarose. Then, the mixture was spread out on 1.5% LB agar to form a double agar layer. The plate was incubated at 37° C. for 18 h to form the plaques. The antiviral activity was defined and calculated as follows: The initial virus titer (NO) and the virus titer after incubation (N) were calculated by counting the plaque number. For each sample, 9 repeats were performed to assess the antiviral activity.

$$Antiviral activity = {log}_{10} ( N / {N}_{0} )$$

### Cell Culture and Cell Viability Measurements

Colorectal adenocarcinoma (HT-29) and Ovarian carcinoma (A2780) cancer cell lines were cultured as monolayers at 37° C. in a 5% CO2 atmosphere, in RPMI 1640 medium, supplemented with 10% Fetal Bovine Serum, 1% Penicillin-Streptomycin, and 1% L-Glutamine. Cytotoxicity was measured by the previously reported MTT method.36 The cells were seeded in a 96-well plate, at a density of ca. 10000 cells per well, and allowed to attach overnight under the conditions mentioned above. The peptides DOPA-Phe-NH2 and DOPA-Phe(4F)—NH2 were dissolved in ethanol to 291 mM and 276 mM, respectively, and then diluted in TDW to a 2 mM concentration. The samples were then serially diluted to create a concentration gradient, with pure TDW as the control, and added to the cells so that the highest concentration was set to 100 PM. The plate was incubated for 24 hours under the same conditions. MTT, 0.1 mg in 20 μl, was added to each well, followed by an additional three-hour incubation. The medium was removed and 200 μl of isopropanol were added to each well, and the absorbance at 550 nm was measured (Spark 10 M multimode microplate reader spectrophotometer, Tecan Group Ltd. Mannedorf, Switzerland). Cell viability was calculated by comparing the formazan absorbance in the treated wells to the untreated control wells. Each measurement was repeated in three wells per plate, and at least on three different days, to total at least 9 repetitions. The relative IC50 values and the standard error of the means were determined by nonlinear regression of a variable slope (four parameters) model, using the GraphPad Prism 5.0 software.

### Antiviral Activity of DOPA-Phe(Br)—OH Against Fruit Viruses

The potential antiviral activity of DOPA-Phe(Br)—OH against fruit viruses and other microbial sources was also investigated. Various brominated peptides, including DOPA-Phe(Br)—OH and DOPA-Phe(Br)—NH2, were tested and found capable of eradicating fruit and plant viruses.

1-57. (canceled)

58. An antimicrobial formulation comprising at least one
antimicrobial peptide of structure (II): DOPA-(AA)ₙ-M, wherein DOPA is 3,4-dihydroxy-L-phenylalanin (DOPA), or a hydroxylated DOPA,
AA is an amino acid or an amino acid sequence or peptide comprising between 2 and 4 amino acids;
n is an integer between 1 and 4; and
M is a functionality or a group of atoms present at the terminal end of the peptide, M being:
(1) —O-M, wherein —O— is an oxygen atom of the amino acid and M is selected from a metal cation (such as Li, Na, K, and other monovalent metal cations); —H; —C₁-C₅alkyl; —C₆-C₁₀aryl; —C(═O)—O—C₁-C₅alkyl; —C(═O)—O—C₆-C₁₀aryl; —C(═O)—O—C₁-C₅alkylene-C₆-C₁₀aryl; —NH₂; —NHR₁, —NHR₁R₂, —NR₁R₂R₃, wherein each of R₁, R₂ and R₃, independently of the other is —H or a —C₁-C₅alkyl; or
(2) and amine or an ammonium selected from —NH₂; —NHR₁, —NHR₁R₂, —NR₁R₂R₃, wherein each of R₁, R₂ and R₃, independently of the other is —H or a —C₁-C₅alkyl.

59. The formulation according to claim 58, wherein the hydroxylated DOPA is hydroxy-DOPA, dihydroxy-DOPA or trihydroxy-DOPA.

60. The formulation according to claim 58, wherein the peptide of structure (II) is DOPA-(AA)ₙ-O-M, wherein each of DOPA, AA, n, O and M is as defined in claim 5

61. The formulation according to claim 58, wherein the peptide of structure (II) is DOPA-(AA)ₙ-NH₂, DOPA-(AA)ₙ-NHR₁, DOPA-(AA)ₙ-NHR₁R₂+, or DOPA-(AA)ₙ-NR₁R₂R₃+, wherein each of R₁, R₂ and R₃, independently of the other is —H or a —C₁-C₅alkyl, and when the nitrogen atom is positively charged, the peptide is associated with at least one counter ion.

62. The formulation according to claim 58, wherein each of the amino acids designated AA is an aromatic amino acid.

63. The formulation according to claim 58, wherein at least one of the amino acids designated AA is an aromatic amino acid.

64. The formulation according to claim 58, wherein at least one of the amino acids designated AA is a brominated or a chlorinated aromatic amino acid.

65. The formulation according to claim 58, wherein the peptide is selected from:
DOPA-(AA)ₙ-O-M;
DOPA-(AA)ₙ-NH₂, DOPA-(AA)ₙ-NHR₁, DOPA-(AA)ₙ-NHR₁R₂+, or DOPA-(AA)n-NR₁R₂R₃+, wherein each of R₁, R₂ and R₃, independently of the other is —H or a —C₁-C₅alkyl;
DOPA-Phe-Phe-OM;
DOPA-Phe-Phe(4Br)-OM;
DOPA-Phe(4Br)-Phe-OM;
DOPA-Phe(4Br)-Phe(4Br)-OM;
DOPA-Phe-OM;
DOPA-Phe-Phe(4Cl)-OM;
DOPA-Phe(4Cl)-Phe-OM;
DOPA-Phe(4Cl)-Phe(4Br)-OM;
DOPA-Phe(4Cl)-Phe(4Cl)-OM;
DOPA-Phe(4Br)-Phe(4Cl)-OM;
DOPA-Phe-OM;
DOPA-Phe-NH₂;
DOPA-Phe(4Br)—NH₂;
DOPA-Phe(4Cl)—NH₂;
DOPA-Phe-Phe-OMe;
DOPA-Phe-Phe(4Br)—OMe;
DOPA-Phe-Phe(4Cl)—OMe;
DOPA-Phe(4Br)-Phe-OMe;
DOPA-Phe(4Br)-Phe(4Br)—OMe;
DOPA-Phe(4Cl)-Phe-OMe;
DOPA-Phe(4Cl)-Phe(4Br)—OMe;
DOPA-Phe(4Cl)-Phe(4Cl)—OMe;
DOPA-Phe(4Br)-Phe(4Br)—OMe;
DOPA-Phe-OMe; and
Cbz-DOPA-Phe-OH.

66. The formulation according to claim 58, comprising a liquid medium and the peptide, the formulation being for forming antimicrobial films on a surface region, or for preventing attack or damage or degradation or decomposition or poisoning due to presence of a microbial infection source, or for application on live plants, on fruits and vegetables or seeds.

67. The formulation according to claim 58 being an agricultural formulation for decreasing microbial load in a liquid medium or a surface.

68. The formulation according to claim 58, for
(i) decreasing microbial load by preventing microbial infection, propagation, attachment or spreading, by eradicating of microbial cells or virions in a target, after they have been established, or
(ii) repelling microbial settling or attachment or assembly on a surface of live plants, pre-harvested or post-harvested fruits, vegetables, flowers and seeds, or
(iii) improving growth, storage, handling, safety, effectiveness of live agricultural products, or
(iv) for preventing spoilage and production of microorganism-derived undesirable by-products.

69. The formulation according to claim 58, for preventing propagation or spreading of microorganism by prevention of assembly and production of new microbial cells.

70. The formulation according to claim 58, formulated as a disinfectant composition or as a preservative.

71. The formulation according to claim 70, being in a form of a paint, a latex emulsion, a polymer emulsion, an adhesive, a sealant, a caulk, a mineral or pigment slurry, a printing ink, a pesticide formulation, a household product, a personal care product, a hygiene product, a metal working fluid, a pharmaceutical, a foodstuff, a food additive, or any packaging material.

72. The formulation according to claim 58, being an antiviral formulation.

73. The formulation according to claim 58, being an antiviral formulation wherein the viral infector is a human or a canine coronavirus.

74. A method being:
a method of eradicating or reducing a population of microorganism in a solid or liquid medium, the method comprises contacting or adding or treating or allowing interaction of said medium with a peptide of structure (II) as defined in claim 58, or a formulation comprising same; or
a method of inducing or endowing antimicrobial properties to a surface region of an object, the method comprises contacting said surface region with a peptide of structure (II) as defined in claim 58 or a formulation comprising same and optionally allowing said formulation to form a solid film of said peptide; or
a method of reducing microbial load on or in an object, the method comprising contacting said object with a peptide of structure (II) as defined in claim 58 or a formulation comprising same and optionally allowing said formulation to form a solid film of said peptide; or
a method or protecting live plants, post-harvest plants, fruits and vegetables, seeds, or seedings from attack by at least one microbial source or pathogen, the method comprising contacting same with a peptide of structure (II) as defined in claim 58 or a formulation comprising the peptide and optionally allowing said formulation to form a solid film of said peptide; or
a method of protecting seeds against microbial attack by a microbial source or a pathogen, the method comprising contacting said seeds with a peptide of structure (II) as defined in claim 58 or a formulation comprising same and optionally allowing said formulation to form a solid film of said peptide.

75. An antiviral reagent comprising a peptide of structure (II) as defined in claim 58, the reagent being for use as a disinfectant.

76. A disinfectant or a sterilizing agent comprising a liquid medium and a peptide of structure(II) as defined in claim 58.

77. An antimicrobial formulation comprising an antimicrobial peptide consisting a peptide of structure (II) as defined in claim 58.
